(19) United States
(12) Patent Application Publication
(10) Pub. No.: US 2020/0281958 A1
(43) Pub. Date: Sep. 10, 2020

(54) STABLE FORMULATIONS FOR THE ORAL ADMINISTRATION OF AMPHOTERICIN B AND RELATED METHODS

(71) Applicant: ICO THERAPEUTICS INC., Vancouver (CA)

(72) Inventor: Peter HNIK, Vancouver (CA)

(21) Appl. No.: 16/653,188

(22) Filed: Oct. 15, 2019

Related U.S. Application Data
(63) Continuation of application No. 15/541,236, filed on Jun. 30, 2017, now abandoned.
(60) Provisional application No. 62/101,746, filed on Jan. 9, 2015.
(60) Provisional application No. 62/101,774, filed on Jan. 9, 2015.

(51) Int. Cl. A61K 31/7048; A61K 45/06; A61K 47/14; A61K 31/7072; A61K 31/513; A61K 31/536; A61K 9/107; A61K 9/00; A61K 39/395; A61K 47/22; A61K 47/24

(52) U.S. Cl. CPC A61K 31/7048; A61K 45/06; A61K 47/14; A61K 31/7072; A61K 31/513; A61K 47/24; A61K 9/1075; A61K 9/0053; A61K 39/3955; A61K 47/22; A61K 31/536

(57) ABSTRACT

The present invention provides oral AmpB and/or protease inhibitor formulations and their use to treat infectious disease, including HIV.

### CROSS-REFERENCE TO RELATED APPLICATIONS

This application is a Continuation Application of U.S. application Ser. No. 15/541,236, filed Jun. 30, 2017, which is a National Stage application under 35 U.S.C. § 371 of International Application No. PCT/US2016/12727, having an International Filing Date of Jan. 8, 2016, which claims priority to U.S. Provisional Application No. 62/101,746, filed on Jan. 9, 2015 and U.S. Provisional Application No. 62/101,774, filed on Jan. 9, 2015, each of which is incorporated by reference herein in its entirety.

### BACKGROUND

### Technical Field

The present invention relates generally to stable formulations of amphotericin B and/or protease inhibitors, and methods of using such formulations for the treatment of diseases, including HIV infection.

### Description of the Related Art

In HIV-1 seropositive individuals being treated with highly active antiretroviral therapy (HAART), virological suppression to levels below the assay detection limit leads to delayed disease progression and reconstitutes the immune system by increasing the peripheral CD4+ T cell count. However, the complete eradication of HIV was not believed possible due to the persistence of the latent HIV reservoir established in resting memory CD4+ T cells and other sites. Studies have suggested that this latent reservoir is the source of viral reactivation. In addition, HIV-1 can latently infect monocytes, also contributing to viral persistence.

Methods for clearing latent HIV reservoirs and thus eradicating HIV-1 infection in a patient are urgently needed. The reactivation of latently infected cells, while at the same time inhibiting HIV replication, may allow the immune system to eradicate infected cells. Attempts have been made over the years to identify compounds capable of reactivating latent HIV-1 in patients.

Amphotericin B has been shown to reactivate HIV-1 replication in monocytic cell lines and in primary macrophages/monocytes. While AmpB did not directly reactivate latently infected T cell lines, their co-culture with primary macrophages showed partial HIV-1 reactivation mediated by AmpB. An AmpB-derivative has been shown to be effective at blocking HIV-1 replication in vitro and inducing T cell activation via the CD3/T cells receptor in HIV-1-infected CD4+ T cells. T cell activation is required for the reactivation of HIV from its latent form, which is essential for elimination of the reservoir. However, despite the fact that AmpB reactivates with the two main cellular reservoirs, i.e., CD4+ T cells and cells from the monocyte-macrophage lineage, it is not known whether this compound could eliminate latent HIV reservoirs without inducing global T cell activation.

Amphotericin B is an effective antifungal agent, and at present, is the drug of choice for treating most serious systemic fungal infections. The drug binds strongly to ergosterol, a major sterol component of fungal membranes, forming pores in the membranes causing disruption of the membrane, cell permeability, and lysis.

Amphotericin B has had limitations in clinical administration due to several unfavorable properties. First, amphotericin B has a strong binding affinity for cholesterol, a sterol present in most mammalian cell membranes, and therefore is capable of disrupting host cells. This leads to renal toxicity of the drug. Second, amphotericin B is not absorbed in the gastrointestinal tract (GIT) due to its poor solubility and its sensitivity to the acid environment of the stomach. To overcome this problem, amphotericin B is used parenterally as liposomal (AMBISOME®) or as colloidal dispersion (FUNGIZONE®, ABELCET®) for the treatment of certain systemic fungal infections (Arikan and Rex, 2001. Lipid-based antifungal agents: current status. Curr. Pharm. Des. 5, 393-415).

However, intravenous injection and infusion of amphotericin B have significant disadvantages. First, the intravenous injection and infusion of amphotericin B has been associated with considerable fluctuation of drug concentrations in the blood and side effects such as nephrotoxicity (Miller et al., 2000, Nanosuspensions for the formulation of poorly soluble drugs-rationale for development and what we can expect for the future. In: Nielloud, F., Marti-Mestres, G. (Eds.), Pharmaceutical emulsions and suspensions. Plenum Press/Marcel Dekker, New York, pp. 383-408). Second, in addition to the high cost, the injection and infusion formulation of amphotericin B have also presented low compliance and technical problems with administration in endemic countries.

Similarly, improved oral formulations of other drugs, including protease inhibitors for the treatment of HIV infection are also desirable.

The present invention overcomes these disadvantages by providing an amphotericin B formulation that can be administered orally, which may be used to eradicate latent HIV, as well as protease inhibitor formulations that may be administered orally to treat HIV infections.

### BRIEF SUMMARY OF THE INVENTION

The present invention provides novel protease inhibitor formulations and related methods of use thereof.

In one embodiment, the present invention provides an oral protease inhibitor formulation comprising:

(a) a protease inhibitor;

(b) one or more fatty acid glycerol esters;

(c) one or more polyethylene oxide-containing fatty acid esters; and

(d) optionally a tocopherol polyethylene glycol succinate.

In a related embodiments, the present invention includes a method of treating an infectious disease, e.g., HIV, in a subject in need thereof, comprising providing the protease inhibitor formulation to the subject.

In a related embodiment, the present invention provides methods of reactivating a latent HIV reservoir in a subject in need thereof, comprising providing to the subject an AmpB formulation described herein.

In particular embodiments, the AmpB formulation comprising:

(a) AmpB;

(b) one or more fatty acid glycerol esters;

(c) one or more polyethylene oxide-containing fatty acid esters; and

(d) optionally a tocopherol polyethylene glycol succinate.

In certain embodiments, the subject is further provided with an immune modulating agent.

In certain embodiments, the present invention includes a method of treating or preventing HIV in a subject in need thereof, comprising providing to the subject a protease inhibitor formulation of the present invention and an AmpB formulation of the present invention. In particular embodiments, the subject is provided with a formulation comprises:

(a) a protease inhibitor;

(b) AmpB

(c) one or more fatty acid glycerol esters;

(d) one or more polyethylene oxide-containing fatty acid esters; and

(e) optionally a tocopherol polyethylene glycol succinate.

In particular embodiments, the formulations are provided orally.

In related embodiments, the present invention includes a protease inhibitor formulation, comprising: a protease inhibitor; one or more fatty acid glycerol esters;

one or more polyethylene oxide-containing phospholipids or one or more polyethylene oxide-containing fatty acid esters; and optionally, a tocopherol polyethylene glycol succinate. In particular embodiments, the protease inhibitor formulation further comprises amphotericin B.

In one particular embodiment, the protease inhibitor formulation comprises: a protease inhibitor; one or more fatty acid glycerol esters; one or more polyethylene oxide-containing phospholipids; and optionally, a tocopherol polyethylene glycol succinate.

In another embodiment, the protease inhibitor formulation comprises: a protease inhibitor; one or more fatty acid glycerol esters; one or more polyethylene oxide-containing fatty acid esters; and optionally, a tocopherol polyethylene glycol succinate.

In particular embodiments, the formulations comprise the tocopherol polyethylene glycol succinate. In certain embodiments, the tocopherol polyethylene glycol succinate is a vitamin E tocopherol polyethylene glycol succinate. In some embodiments, the tocopherol polyethylene glycol succinate is present in the formulation in an amount from about 0.1 to about 10 percent by volume based on the total volume of the formulation.

In particular embodiments of any of the formulations, the protease inhibitor is selected from the group consisting of: amprenavir, ritonavir, saquinavir, tipranavir, atazanavir, fosamprenavir, lopinavir, indinavir, darunavir, and nelfinavir.

In certain embodiments of any of the formulations, amphotericin B is present in the formulation in an amount from about 0.5 to about 10 mg/mL of the formulation.

In certain embodiments of formulations, the fatty acid glycerol esters comprise from about 32 to about 52% by weight fatty acid monoglycerides. In certain embodiments, the fatty acid glycerol esters comprise from about 30 to about 50% by weight fatty acid diglycerides. In certain embodiments, the fatty acid glycerol esters comprise from about 5 to about 20% by weight fatty acid triglycerides.

In particular embodiments of formulations, the polyethylene oxide-containing phospholipids comprise a C8-C22 saturated fatty acid ester of a phosphatidyl ethanolamine polyethylene glycol salt. In certain embodiments, the polyethylene oxide-containing phospholipids comprise a distearoylphosphatidyl ethanolamine polyethylene glycol salt. In certain embodiments, the distearoylphosphatidyl ethanolamine polyethylene glycol salt is selected from the group consisting of a distearoylphosphatidyl ethanolamine polyethylene glycol 350 salt, a distearoylphosphatidyl ethanolamine polyethylene glycol 550 salt, a distearoylphosphatidyl ethanolamine polyethylene glycol 750 salt, a distearoylphosphatidyl ethanolamine polyethylene glycol 1000 salt, distearoylphosphatidyl ethanolamine polyethylene glycol 2000 salt, and mixtures thereof.

In particular embodiments of formulations, the polyethylene oxide-containing fatty acid esters comprise a polyethylene oxide ester of a C8-C22 saturated fatty acid. In certain embodiments, the polyethylene oxide-containing fatty acid esters comprise a polyethylene oxide ester of a C12-C18 saturated fatty acid. In some embodiments, the polyethylene oxide-containing fatty acid esters comprise a polyethylene oxide having an average molecular weight of from about 750 to about 2000. In some embodiments, the ratio of the fatty acid glycerol esters to polyethylene oxide-containing fatty acid esters is about 60:40 v/v or about 50:50 v/v. In certain embodiments, the polyethylene oxide-containing fatty acid esters is selected from the group consisting of lauric acid esters, palmitic acid esters, stearic acid esters, and mixtures thereof.

In particular embodiments of the invention, the protease inhibitor formulations comprise two or more protease inhibitors.

In particular embodiments of the invention, the protease inhibitor formulations comprise one or more additional therapeutic agents. In certain embodiments, the one or more additional therapeutic agents is an immune modulating agent. In some embodiments, the immune modulating agent is an anti-PD-1 antibody agent or an anti-CTLA-4 agent.

In particular embodiments of any of the formulations, the formulation is a self-emulsifying drug delivery system.

In a further embodiment, the present invention includes a method of treating or preventing an infectious disease in a subject in need thereof, comprising providing to the subject the protease inhibitor formulation of the present invention. In some embodiments, the protease inhibitor formulation is provided to the subject orally or topically. In some embodiments, the infectious disease is human immunodeficiency virus type 1 (HIV-1) infection or acquired immune deficiency syndrome (AIDS). In some embodiments, the infectious disease is a protozoal infection. In some embodiments, the protease inhibitor formulation is provided to the subject at least once a day, at least once every two days, or at least once a week, for a period of time. In certain embodiments, the protease inhibitor formulation is provided to the subject in combination with one or more additional therapeutic agents. In particular embodiments, the one or more additional therapeutic agents is selected from AmpB, HAART, or an immune modulating agent. In some embodiments, the immune modulating agent is an anti-PD-1 antibody agent or an anti-CTLA-4 agent.

In another related embodiment, the present invention includes a method of reactivating a latent HIV reservoir in a subject in need thereof, comprising providing to the subject an amphotericin B (AmpB) formulation comprising: AmpB; one or more fatty acid glycerol esters; one or more polyethylene oxide-containing phospholipids or one or more polyethylene oxide-containing fatty acid esters; and optionally, a tocopherol polyethylene glycol succinate. In some embodiments, the AmpB formulation further comprises a protease inhibitor. In particular embodiments, the AmpB formulation comprises: AmpB; one or more fatty acid glycerol esters; one or more polyethylene oxide-containing phospholipids; and

optionally, a tocopherol polyethylene glycol succinate. In some embodiments, the AmpB formulation comprises: AmpB; one or more fatty acid glycerol esters; one or more polyethylene oxide-containing fatty acid esters; and optionally, a tocopherol polyethylene glycol succinate. In particular embodiments of any of the formulations, the formulation comprises the tocopherol polyethylene glycol succinate. In some embodiments, the tocopherol polyethylene glycol succinate is a vitamin E tocopherol polyethylene glycol succinate. In certain embodiments, the tocopherol polyethylene glycol succinate is present in the formulation in an amount from about 0.1 to about 10 percent by volume based on the total volume of the formulation. In some embodiments, the protease inhibitor is selected from the group consisting of: amprenavir, ritonavir, saquinavir, tipranavir, atazanavir, fosamprenavir, lopinavir, indinavir, darunavir, and nelfinavir. In some embodiments, amphotericin B is present in the formulation in an amount from about 0.5 to about 10 mg/mL of the formulation.

In certain embodiments when present, the fatty acid glycerol esters comprise from about 32 to about 52% by weight fatty acid monoglycerides. In some embodiments, the fatty acid glycerol esters comprise from about 30 to about 50% by weight fatty acid diglycerides. In some embodiments, the fatty acid glycerol esters comprise from about 5 to about 20% by weight fatty acid triglycerides.

In certain embodiments when present, the polyethylene oxide-containing phospholipids comprise a C8-C22 saturated fatty acid ester of a phosphatidyl ethanolamine polyethylene glycol salt. In some embodiments, the polyethylene oxide-containing phospholipids comprise a distearoylphosphatidyl ethanolamine polyethylene glycol salt. In certain embodiments, the distearoylphosphatidyl ethanolamine polyethylene glycol salt is selected from the group consisting of a distearoylphosphatidyl ethanolamine polyethylene glycol 350 salt, a distearoylphosphatidyl ethanolamine polyethylene glycol 550 salt, a distearoylphosphatidyl ethanolamine polyethylene glycol 750 salt, a distearoylphosphatidyl ethanolamine polyethylene glycol 1000 salt, distearoylphosphatidyl ethanolamine polyethylene glycol 2000 salt, and mixtures thereof.

In certain embodiments when present, the polyethylene oxide-containing fatty acid esters comprise a polyethylene oxide ester of a C8-C22 saturated fatty acid. In some embodiments, the polyethylene oxide-containing fatty acid esters comprise a polyethylene oxide ester of a C12-C18 saturated fatty acid. In some embodiments, the polyethylene oxide-containing fatty acid esters comprise a polyethylene oxide having an average molecular weight of from about 750 to about 2000. In some embodiments, the ratio of the fatty acid glycerol esters to polyethylene oxide-containing fatty acid esters is about 60:40 v/v or about 50:50 v/v. In some embodiments, the polyethylene oxide-containing fatty acid ester is selected from the group consisting of lauric acid esters, palmitic acid esters, stearic acid esters, and mixtures thereof.

In certain embodiments of the methods and formulations of the present invention, an AmpB formulation or protease inhibitor formulation comprises one or more additional therapeutic agents. In some embodiments, the one or more additional therapeutic agents is an immune modulating agent. In certain embodiments, the immune modulating agent is an anti-PD-1 antibody agent or an anti-CTLA-4 agent. In some embodiments, the one or more additional therapeutic agents is an anti-human immunodeficiency virus (HIV) agent. In some embodiments, the anti-HIV agent is a component of HAART. In some embodiments, the anti-HIV agent is a protease inhibitor.

In certain embodiments of the methods and formulations of the present invention, the AmpB formulation or protease inhibitor formulation is a self-emulsifying drug delivery system.

In particular embodiments of any of the methods of the present invention, the AmpB formulation or protease inhibitor formulation is provided to the subject orally or topically.

In particular embodiments of the present invention, the subject has been diagnosed with latent human immunodeficiency virus type 1 (HIV-1) infection or acquired immune deficiency syndrome (AIDS).

In particular embodiments, the AmpB formulation or protease inhibitor formulation is provided to the subject at least once a day, at least once every two days, or at least once a week, for a period of time, e.g., a month or greater, two months or greater, four months or greater, six months or greater, one year or greater, two years or greater, five years or greater, 10 years or greater, or for a lifetime.

### BRIEF DESCRIPTION OF THE DRAWINGS

FIG. 1 is a flowchart of the study showing the steps of the experimental procedure: the sequence of purification steps, assays and analyses performed with leukapheresis samples from HIV infected individuals.

FIG. 2 is a graph showing HIV proviral DNA transcripts per million in immune cells as determined by qPCR with samples from 8 HIV-infected subjects (ICO-ST1 to ICO-ST8) on HAART. qPCR was performed on DNA of ax106 PBMC and purified populations of memory CD4+ T cells, CD8+ T cells, and monocytes isolated from patient PBMC. The assay detection limit was set at 3 DNA copies/10⁶ cells. Bars are means of triplicate wells and show the relative HIV DNA copies per 10⁶ cells. For each subject, the bars from left to right correspond to: PBMC, sorted memory CD4+ T cells, sorted total CD8+ T cells, and CD14+ monocytes. Black dots represent the values of HIV DNA copies for each of the triplicate measurements.

FIG. 3 provides graphs showing viral production as measured by ultrasensitive RT-PCR in cell culture supernatants of memory CD4+ T cells isolated from subjects ICO-ST2 (responder: viral production>10 HIV RNA copies per 106 sorted memory CD4 T cells; left) and ICO-ST7 (non-responder: viral production<1 HIV RNA copies per 10⁶ sorted memory CD4 T cells; right) cultured in the presence or absence of different concentrations of oral Amp-B or with antibodies to CD3/CD28 for 6 days in the presence of HAART. All RT-PCR reactions were performed in duplicate for each of the three biological replicates. The assay detection limit was set at a copy of HIV RNA. Bars are means of sixplicate wells and show the relative viral production defined by the number of HIV RNA copies per 10⁶ cells. Dots represent the values of viral production obtained in the presence of different compounds for each of the sixplicate measurements. For each of the two subjects, the bars from left to rights indicate: Amp-B at 0 uM; Amp-B at 0.04 uM; Amp-B at 0.2 uM, Amp-B at 1 uM, and anti-CD3/antiCD28.

FIG. 4 is a graph showing the effect of increasing concentrations of oral Amp-B on viral production in memory CD4+ T cells from all recruited subjects cultured in the presence or absence of different concentrations of oral Amp-B or with antibodies to CD3/CD28 for 6 days in the presence of HAART. HIV-infected subjects were defined as very low or low responders when viral production was >1 or >10 HIV RNA copies per 10⁶ sorted memory CD4 T cells, respectively. HIV-infected subjects were defined as non-responders when viral production was under the assay detection limit of <1 HIV RNA Copies per 10⁶ sorted memory CD4 T cells. Bars are mean of viral production obtained from all subjects and all replicates. The bars from left to right indicate: Amp-B at 0 uM; Amp-B at 0.04 uM; Amp-B at 0.2 uM, Amp-B at 1 uM, and anti-CD3/antiCD28.

FIG. 5 is a graph showing the effect of increasing concentrations of oral Amp-B on viral production in CD14+ monocytes from all recruited subjects cultured in the presence or absence of different concentrations of oral Amp-B or with antibodies to CD3/CD28 for 6 days in the presence of HAART. HIV-infected subjects were defined as very low or low responders when viral production was >1 or >10 HIV RNA copies per 10⁶ sorted memory CD4 T cells, respectively. HIV-infected subjects were defined as non-responders when viral production was under the assay detection limit of <1 HIV RNA Copies per 10⁶ sorted memory CD4 T cells. Bars are mean of viral production obtained from all subjects and all replicates. The bars from left to right indicate: Amp-B at 0 uM; Amp-B at 0.04 uM; Amp-B at 0.2 uM, Amp-B at 1 uM, and anti-CD3/antiCD28. *The numbers across the top show the frequency of patients responsive to treatment for each condition tested.

FIGS. 6A-6C are graphs showing the effect of oral Amp-B on the size of the viral reservoir in memory CD4+ T cells from seropositive subjects. FIGS. 6A, 6B and 6C show the quantification of integrated HIV DNA by ultrasensitive real time PCR after 6 days of co-culture for ICO-ST2, ICO-ST7 and all combined subjects, respectively. In FIG. 6C, bars are means of the number of HIV integrated DNA per 106 sorted memory CD4+ T cells obtained from all subjects and all replicates. Dots represent the values of HIV DNA obtained in the presence of different compounds, which from left to right indicate: Amp-B at 0 uM; Amp-B at 0.04 uM; Amp-B at 0.2 uM, Amp-B at 1 uM, and anti-CD3/antiCD28.

FIGS. 7A and 7B are graphs showing the effect of oral Amp-B on T cell counts in subject ICO-ST2 (FIG. 7A) or subject ICO-ST7 (FIG. 7B) following stimulation and co-culture with varying concentration of oral Amp-B. For each cell type, PBMC or mCD4/TCD8 cocultures, the bars from left to right indicate: Amp-B at 0 uM; Amp-B at 0.04 uM; Amp-B at 0.2 uM, and Amp-B at 1 uM. Cocultures were performed in triplicate. CD4/TCD8 indicates a mix of sorted memory CD4+ T cells and sorted total CD8+ T cells at a ratio of 2:1.

FIGS. 8A-8D are graphs showing the percentage change of the CD4+(FIGS. 8A and 8C) and the CD8+(FIGS. 8B and 8D) T cell counts at each oral Amp-B concentration for the mean of triplicates in all eight patients in PBMC (top panels) and in the memory CD4+T cell/Total CD8+ T cells (bottom panels). For each graph, the bars from left to right indicate: Amp-B at 0.04 uM; Amp-B at 0.2 uM, and Amp-B at 1 uM.

FIGS. 9A-9D are graphs showing the percentage change in the cellular counts of CD4+ central memory subset (FIGS. 9A and 9C) and the CD8+ T cell central memory subset (FIGS. 9B and 9D) at each compound concentration using the mean of triplicates in all eight patients in PBMC (Top panel) and in the memory CD4+ T cells/total CD8+ T cell co-cultures (Bottom panel). For each graph, the bars from left to right indicate: Amp-B at 0.04 uM; Amp-B at 0.2 uM, and Amp-B at 1 uM.

FIGS. 10A and 10B are graphs showing the percentage change in the counts of (FIG. 10A) total CD4+ and (FIG. 10B) central memory CD4+ T cells expressing or not PD-1 at each oral Amp-B concentration tested compared to the 0 uM control using the mean of triplicate values obtained from memory CD4+/total CD8+ T cell co-cultures. A Wilcoxon-signed rank test was used to evaluate whether differences to the control were statistically significant (p<0.05).

FIGS. 11A and 11B are graphs showing the functional profile of HIV-specific proliferating CD4+ T Cells in presence of increasing concentration of oral Amp-B in sorted memory CD4+ T cell/total CD8+ T cells in (FIG. 11A) virological responder (ICO-ST2) and (FIG. 11B) non-responder (ICO-ST7). Response profiles were generated using Boolean analysis of three functional response gates, resulting in eight separate functional T cell subsets. For each condition, the bars from left to right indicate: Amp-B at 0 uM; Amp-B at 0.04 uM; Amp-B at 0.2 uM, and Amp-B at 1 uM.

FIGS. 12A and 12B are graphs showing functional profile of HIV-specific proliferating CD8+ T Cells in presence of increasing concentration of oral Amp-B in sorted memory CD4+/total CD8+ T cells in (FIG. 12A) virological responder (ICO-ST2) and (FIG. 12B) non-responder (ICO-ST7). Response profiles were generated using Boolean analysis of three functional response gates, resulting in eight separate functional T cell subsets. For each condition, the bars from left to right indicate: Amp-B at 0 uM; Amp-B at 0.04 uM; Amp-B at 0.2 uM, and Amp-B at 1 uM. Cocultures were performed in triplicate.

FIG. 13 is a graph showing increased concentration of oral Amp-B induced significant toxicity on stimulated PBMC and sorted memory CD4+/Total CD8+ T cells. For each of the four cells/condition, the bars from left to right indicate: Amp-B at 0 uM; Amp-B at 0.04 uM; Amp-B at 0.2 uM, and Amp-B at 1 uM.

### DETAILED DESCRIPTION OF THE INVENTION

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (3rd Edition, 2000); DNA Cloning: A Practical Approach, vol. I & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R. I. (2005) Culture of Animal Cells, a Manual of Basic Technique, 5^{th} Ed. Hoboken N.J., John Wiley & Sons; B. Perbal, A Practical Guide to Molecular Cloning (3rd Edition 2010); Farrell, R., RNA Methodologies: A Laboratory Guide for Isolation and Characterization (3^{rd} Edition 2005). Poly(ethylene glycol), Chemistry and Biological Applications, ACS, Washington, 1997; Veronese, F., and J. M. Harris, Eds., Peptide and protein PEGylation, Advanced Drug Delivery Reviews, 54(4) 453-609 (2002); Zalipsky, S., et al., “Use of functionalized Poly(Ethylene Glycols) for modification of polypeptides” in Polyethylene Glycol Chemistry: Biotechnical and Biomedical Applications.

All publications, patents and patent applications cited herein are hereby incorporated by reference in their entirety.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “effective amount,” when used in connection with an amphotericin B formulation of the invention or treatment or inhibition of disease or disorder, refers to an amount of the amphotericin B formulation that is useful to treat or inhibit the disease or disorder. The “effective amount” can vary depending upon the mode of administration, specific locus of the disease, the age, body weight, and general health of the subject being treated.

The term “modulating” includes “increasing,” “enhancing” or “stimulating,” as well as “decreasing” or “reducing,” typically in a statistically significant or a physiologically significant amount as compared to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount produced by no composition (e.g., in the absence of any of the YRS polypeptides of the invention) or a control composition, sample or test subject. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount produced by no composition (the absence of an agent or compound) or a control composition, including all integers in between. As one non-limiting example, a control in comparing canonical and non-canonical activities could include the YRS polypeptide of interest compared to a corresponding un-YRS polypeptide. Other examples of “statistically significant” amounts will be apparent from the description provided herein.

A “subject,” as used herein, includes any animal that exhibits a symptom, or is at risk for exhibiting a symptom, which can be treated or diagnosed with an amphotericin B formulation of the invention. Suitable subjects (patients) include laboratory animals (such as mouse, rat, rabbit, or guinea pig), farm animals, and domestic animals or pets (such as a cat or dog). Non-human primates and, preferably, human patients, are included.

“Substantially” or “essentially” means nearly totally or completely, for instance, 95% or greater of some given quantity.

“Treatment” or “treating,” as used herein, includes any desirable effect on the symptoms or pathology of a disease or condition, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

Stable Oral Formulations

The present invention provides oral formulations or AmpB and/or protease inhibitors, which may be used to treat or prevent various diseases or conditions, such as infections, e.g., HIV infection. In particular embodiments, the oral formulations of the present invention are advantageous is delivering the active agent, e.g., protease inhibitor or AmpB, to the lymphatic tissue or brain tissue, as compared to previous formulations of the same active agent. In addition, the oral formulations of the present invention may be used to deliver an active agent, e.g., HAART, across the blood brain barrier at a higher level or rate than prior formulations. Accordingly, delivery of an active agent in a formulation of the present invention these oral formulations can result in increased or higher levels of the active agent in the subject's lymphatic tissue or brain tissue as compared to when the same amount of the active agent is delivered in a different formulation. Consequently, an equivalent amount or level of the active agent in lymphatic tissue or brain tissue may be achieved by delivering a lower dose of the active agent in a formulation of the present invention as compared to other formulations. In certain situations, this may result in decreased undesirable side effects, including interaction with other drugs, or resistance to the active agent.

Amphotericin B Formulations

In one aspect, the present invention provides amphotericin B formulations, methods for making the formulations, methods for administering amphotericin B using the formulations, and methods for treating diseases treatable by amphotericin B by administering the formulations.

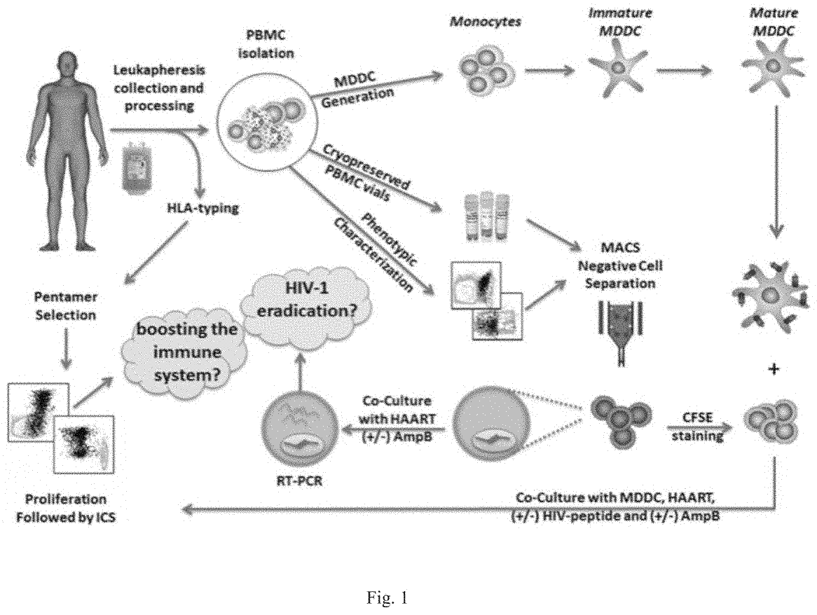

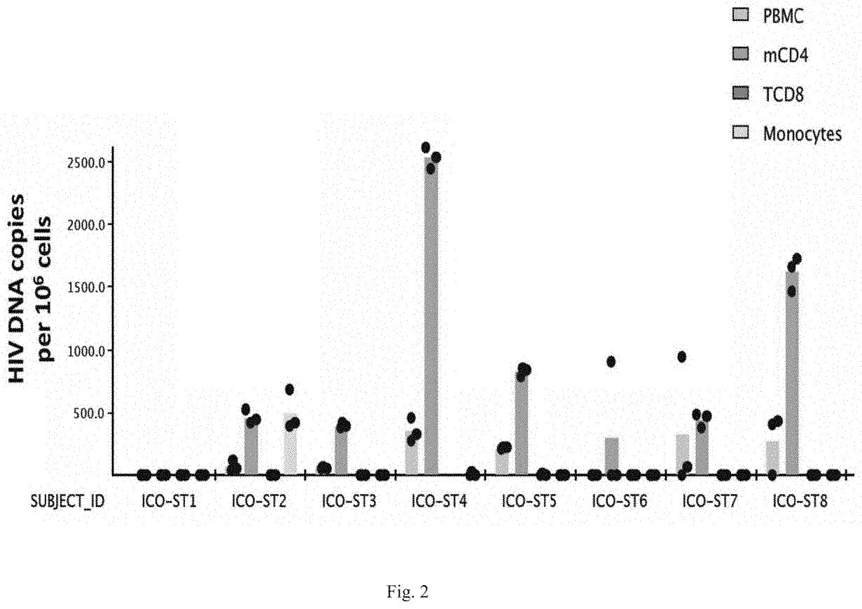

Amphotericin B is an antimycotic polyene antibiotic obtained from Streptomyces nodosus M4575. Amphotericin B is designated chemically as [1R-(1R*,3S*,5R*,6R*,9R*,11R*,15S*,16R*,17R*,18S*,19E,21E,23E,25E, 27E,29E,31 E,33R*,35S*,36R*,37S)]-33-[(3-amino-3,6-dideoxy-β-D-mannopyranosyl) oxy] 1,3,5,6,9,11, 17,37-octahydroxy-15,16,18-trimethyl-13-oxo-14,39-dioxabicyclo-[33.3.1] nonatriaconta-19,21, 23,25,27,29,3 1-heptaene-36-carboxylic acid. The chemical structure of amphotericin B is shown in PCT application Publication Nos. WO 2008/144888 and WO 2011/050457, and in FIG. 1A of U.S. Pat. No. 8,592,382. Crystalline amphotericin B is insoluble in water.

In certain embodiment, the amphotericin formulations of the invention comprise:

(a) amphotericin B;

(b) one or more fatty acid glycerol esters; and

(c) one or more polyethylene oxide-containing phospholipids or one or more polyethylene oxide-containing fatty acid esters.

In another aspect, the present invention provides amphotericin B formulations that comprise:

(a) amphotericin B;

(b) one or more fatty acid glycerol esters;

(c) one or more polyethylene oxide-containing phospholipids or one or more polyethylene oxide-containing fatty acid esters; and

(d) optionally a tocopherol polyethylene glycol succinate.

In representative formulations of any of the formulations described herein, amphotericin B is present in an amount from about 0.5 to about 10 mg/mL, about 0.1 to about 1000 mg/mL, about 0.1 to about 100 mg/mL, about 0.5 to about 50 mg/mL, or about 0.5 to about 20 mg/mL of the formulation. In one embodiment, amphotericin B or pharmaceutically acceptable salt thereof is present in the formulation in about 5 mg/mL or about 10 mg/mL or about 1 mg/mL or about 20 mg/mL. In one embodiment, amphotericin B or its pharmaceutically acceptable salt thereof is present in the formulation in about 7 mg/mL.

In particular embodiments, the amphotericin B formulations include one or more fatty acid glycerol esters, and typically, a mixture of fatty acid glycerol esters. As used herein the term “fatty acid glycerol esters” refers to esters formed between glycerol and one or more fatty acids including mono-, di-, and tri-esters (i.e., glycerides). Suitable fatty acids include saturated and unsaturated fatty acids having from eight (8) to twenty-two (22) carbons atoms (i.e., C8-C22 fatty acids). In certain embodiments, suitable fatty acids include C12-C18 fatty acids.

The fatty acid glycerol esters useful in the formulations can be provided by commercially available sources. A representative source for the fatty acid glycerol esters is a mixture of mono-, di-, and triesters commercially available as PECEOL® (Gattefosse, Saint Priest Cedex, France), commonly referred to as “glyceryl oleate” or “glyceryl monooleate.” When PECEOL® is used as the source of fatty acid glycerol esters in the formulations, the fatty acid glycerol esters comprise from about 32 to about 52% by weight fatty acid monoglycerides, from about 30 to about 50% by weight fatty acid diglycerides, and from about 5 to about 20% by weight fatty acid triglycerides. The fatty acid glycerol esters comprise greater than about 60% by weight oleic acid (C18:1) mono-, di-, and triglycerides. Other fatty acid glycerol esters include esters of palmitic acid (C16) (less than about 12%), stearic acid (C18) (less than about 6%), linoleic acid (C18:2) (less than about 35%), linolenic acid (C18:3) (less than about 2%), arachidic acid (C20) (less than about 2%), and eicosenoic acid (C20:1) (less than about 2%). PECEOL® can also include free glycerol (typically about 1%). In one embodiment, the fatty acid glycerol esters comprise about 44% by weight fatty acid monoglycerides, about 45% by weight fatty acid diglycerides, and about 9% by weight fatty acid triglycerides, and the fatty acid glycerol esters comprise about 78% by weight oleic acid (C18:1) mono-, di-, and triglycerides. Other fatty acid glycerol esters include esters of palmitic acid (C16) (about 4%), stearic acid (Cl 8) (about 2%), linoleic acid (Cl 8:2) (about 12%), linolenic acid (C18:3) (less than 1%), arachidic acid (C20) (less than 1%), and eicosenoic acid (C20:1) (less than 1%).

In certain embodiments, the formulations of the invention can include glycerol in an amount less than about 10% by weight.

In certain embodiments, the formulations include a tocopherol polyethylene glycol succinate.

Amphotericin B Formulations: Polyethylene Oxide-Containing Phospholipids (DSPE-PEGs)

In certain embodiments, the amphotericin B formulations include one or more polyethoxylated lipids. In one embodiment, the polyethoxylated lipids are polyethylene oxide-containing phospholipids, or a mixture of polyethylene oxide-containing phospholipids. In another embodiment, the polyethoxylated lipids are polyethylene oxide-containing fatty acid esters, or a mixture of polyethylene oxide-containing fatty acid esters.

Accordingly, in one embodiment, the amphotericin B formulations of the invention comprise:

(a) amphotericin B;

(b) one or more fatty acid glycerol esters; and

(c) one or more polyethylene oxide-containing phospholipids.

As used herein, the term “polyethylene oxide-containing phospholipid” refers to a phospholipid that includes a polyethylene oxide group (i.e., polyethylene glycol group) covalently coupled to the phospholipid, typically through a carbamate or an ester bond. Phospholipids are derived from glycerol and can include a phosphate ester group and two fatty acid ester groups. Suitable fatty acids include saturated and unsaturated fatty acids having from eight (8) to twenty-two (22) carbons atoms (i.e., C8-C22 fatty acids). In certain embodiments, suitable fatty acids include saturated C 12-Cl 8 fatty acids. Representative polyethylene oxide-containing phospholipids include C8-C22 saturated fatty acid esters of a phosphatidyl ethanolamine polyethylene glycol salt. In certain embodiments, suitable fatty acids include saturated C 12-Cl 8 fatty acids.

The molecular weight of the polyethylene oxide group of the polyethylene oxide-containing phospholipid can be varied to optimize the solubility of the therapeutic agent (e.g., amphotericin B) in the formulation. Representative average molecular weights for the polyethylene oxide groups can be from about 200 to about 5000 (e.g., PEG 200 to PEG 5000).

In one embodiment, the polyethylene oxide-containing phospholipids are distearoyl phosphatidyl ethanolamine polyethylene glycol salts. Representative distearoylphosphatidyl ethanolamine polyethylene glycol salts include distearoylphosphatidyl ethanolamine polyethylene glycol 350 (DSPE-PEG-350) salts, distearoylphosphatidyl ethanolamine polyethylene glycol 550 (DSPE-PEG-550) salts, distearoylphosphatidyl ethanolamine polyethylene glycol 750 (DSPE-PEG-750) salts, distearoylphosphatidyl ethanolamine polyethylene glycol 1000 (DSPE-PEG-1000) salts, distearoylphosphatidyl ethanolamine polyethylene glycol 1500 (DSPE-PEG-1500) salts, and distearoylphosphatidyl ethanolamine polyethylene glycol 2000 (DSPE-PEG-2000) salts. Mixtures can also be used. For the distearoylphosphatidyl ethanolamine polyethylene glycol salts above, the number (e.g., 350, 550, 750, 1000, and 2000) designates the average molecular weight of the polyethylene oxide group. The abbreviations for these salts used herein is provided in parentheses above.

Suitable distearoylphosphatidyl ethanolamine polyethylene glycol salts include ammonium and sodium salts. The chemical structure of distearoylphosphatidyl ethanolamine polyethylene glycol 2000 (DSPE-PEG-2000) ammonium salt is shown in FIG. 1B of U.S. Pat. No. 8,592,382, which shows that the polyethylene oxide-containing phospholipid includes a phosphate ester group and two fatty acid ester (stearate) groups, and a polyethylene oxide group covalently coupled to the amino group of the phosphatidyl ethanolamine through a carbamate bond.

As noted above, the polyethylene oxide-containing phospholipid affects the ability of the formulation to solubilize a therapeutic agent. In general, the greater the amount of polyethylene oxide-containing phospholipid, the greater the solubilizing capacity of the formulation for difficultly soluble therapeutic agents. The polyethylene oxide-containing phospholipid can be present in the formulation in an amount from about 1 mM to about 30 mM based on the volume of the formulation. In certain embodiments, the distearoylphosphatidyl ethanolamine polyethylene glycol salt is present in the formulation in an amount from 1 mM to about 30 mM based on the volume of the formulation. In one embodiment, the distearoylphosphatidyl ethanolamine polyethylene glycol salt is present in the formulation in about 15 mM based on the volume of the formulation.

In one embodiment, the amphotericin B formulations of the invention comprise:

(a) amphotericin B;

(b) oleic acid glycerol esters; and

(c) a distearoylphosphatidyl ethanolamine polyethylene glycol salt.

In one embodiment, the amphotericin B formulation of the invention includes amphotericin B, PECEOL®, and a distearoylphosphatidyl ethanolamine polyethylene glycol salt. In this embodiment, the distearoylphosphatidyl ethanolamine polyethylene glycol salt is present in an amount up to about 30 mM. The preparation and characterization of representative amphotericin B formulations of the invention that include polyethylene oxide-containing phospholipids is described in PCT Publication No. WO2008/144888.

In certain embodiments, the amphotericin B formulations that include polyethylene oxide-containing phospholipids include amphotericin B that is both partially solubilized (dissolved) and present as solid particles to provide a fine solid dispersion. Dispersion of the formulation in aqueous media provides a nano-/microemulsion having emulsion droplets that range in size from about 50 nm to about 5 μm.

Amphotericin B Formulations: Polyethylene Oxide-Containing Fatty Acid Esters

In certain embodiments, the amphotericin B formulations include one or more polyethoxylated lipids such as polyethylene oxide-containing phospholipids or one or more polyethylene oxide-containing fatty acid esters, and typically, a mixture of polyethylene oxide-containing phospholipids or a mixture of polyethylene oxide-containing fatty acid esters.

Accordingly, in one embodiment, the amphotericin B formulations of the invention comprise:

(a) amphotericin B;

(b) one or more fatty acid glycerol esters; and

(c) one or more polyethylene oxide-containing fatty acid esters.

In another embodiment, the amphotericin B formulations of the invention comprise:

(a) amphotericin B;

(b) one or more fatty acid glycerol esters;

(c) one or more polyethylene oxide-containing fatty acid esters; and

(d) optionally a tocopherol polyethylene glycol succinate.

As used herein, the term “polyethylene oxide-containing fatty acid ester” refers to a fatty acid ester that includes a polyethylene oxide group (i.e., polyethylene glycol group) covalently coupled to the fatty acid through an ester bond. Polyethylene oxide-containing fatty acid esters include mono- and di-fatty acid esters of polyethylene glycol. Suitable polyethylene oxide-containing fatty acid esters are derived from fatty acids including saturated and unsaturated fatty acids having from eight (8) to twenty-two (22) carbons atoms (i.e., a polyethylene oxide ester of a C8-C22 fatty acid). In certain embodiments, suitable polyethylene oxide-containing fatty acid esters are derived from fatty acids including saturated and unsaturated fatty acids having from twelve (12) to eighteen (18) carbons atoms (i.e., a polyethylene oxide ester of a C 12-Cl 8 fatty acid). Representative polyethylene oxide-containing fatty acid esters include saturated C8-C22 fatty acid esters. In certain embodiments, suitable polyethylene oxide-containing fatty acid esters include saturated C 12-Cl 8 fatty acids.

The molecular weight of the polyethylene oxide group of the polyethylene oxide-containing fatty acid ester can be varied to optimize the solubility of the therapeutic agent (e.g., amphotericin B) in the formulation. Representative average molecular weights for the polyethylene oxide groups can be from about 350 to about 2000. In one embodiment, the average molecular weight for the polyethylene oxide group is about 1500.

In this embodiment, the amphotericin B formulations include one or more polyethylene oxide-containing fatty acid esters, and typically, a mixture of polyethylene oxide-containing fatty acid esters (mono- and di-fatty acid esters of polyethylene glycol). The polyethylene oxide-containing fatty acid esters useful in the formulations can be provided by commercially available sources. Representative polyethylene oxide-containing fatty acid esters (mixtures of mono- and diesters) are commercially available under the designation GELUCIRE® (Gattefosse, Saint Priest Cedex, France). Suitable polyethylene oxide-containing fatty acid esters can be provided by GELUCIRE® 44/14, GELUCIRE® 50/13, and GELUCIRE® 53/10. The numerals in these designations refer to the melting point and hydrophilic/lipophilic balance (HLB) of these materials, respectively. GELUCIRE® 44/14, GELUCIRE® 50/13, and GELUCIRE® 53/10 are mixtures of (a) mono-, di-, and triesters of glycerol (glycerides) and (b) mono- and diesters of polyethylene glycol (macrogols). The GELUCIRES can also include free polyethylene glycol (e.g., PEG 1500).

Laurie acid (C 12) is the predominant fatty acid component of the glycerides and polyethylene glycol esters in GELUCIRE® 44/14. GELUCIRE® 44/14 is referred to as a mixture of glyceryl dilaurate (lauric acid diester with glycerol) and PEG dilaurate (lauric acid diester with polyethylene glycol), and is commonly known as PEG-32 glyceryl laurate (Gattefosse) lauroyl macrogol-32 glycerides EP, or lauroyl polyoxylglycerides USP/NF. GELUCIRE® 44/14 is produced by the reaction of hydrogenated palm kernel oil with polyethylene glycol (average molecular weight 1500). GELUCIRE® 44/14 includes about 20% mono-, di- and, triglycerides, about 72% mono- and di-fatty acid esters of polyethylene glycol 1500, and about 8% polyethylene glycol 1500.

GELUCIRE® 44/14 includes lauric acid (C 12) esters (30 to 50%), myristic acid (C14) esters (5 to 25%), palmitic acid (C16) esters (4 to 25%), stearic acid (C18) esters (5 to 35%), caprylic acid (C8) esters (less than 15%), and capric acid (ClO) esters (less than 12%). GELUCIRE® 44/14 may also include free glycerol (typically less than about 1%). In a representative formulation, GELUCIRE® 44/14 includes lauric acid (C12) esters (about 47%), myristic acid (C14) esters (about 18%), palmitic acid (C16) esters (about 10%), stearic acid (C18) esters (about 11%), caprylic acid (C8) esters (about 8%), and capric acid (ClO) esters (about 12%).

Palmitic acid (C16) (40-50%) and stearic acid (C18) (48-58%) are the predominant fatty acid components of the glycerides and polyethylene glycol esters in GELUCIRE® 50/13. GELUCIRE® 50/13 is known as PEG-32 glyceryl palmitostearate (Gattefosse), stearoyl macrogolglycerides EP, or stearoyl polyoxylglycerides USP/NF). GELUCIRE® 50/13 includes palmitic acid (Cl 6) esters (40 to 50%), stearic acid (C 18) esters (48 to 58%) (stearic and palmitic acid esters greater than about 90%), lauric acid (C12) esters (less than 5%), myristic acid (C14) esters (less than 5%), caprylic acid (C8) esters (less than 3%), and capric acid (ClO) esters (less than 3%). GELUCIRE® 50/13 may also include free glycerol (typically less than about 1%). In a representative formulation, GELUCIRE® 50/13 includes palmitic acid (C16) esters (about 43%), stearic acid (C 18) esters (about 54%) (stearic and palmitic acid esters about 97%), lauric acid (C 12) esters (less than 1%), myristic acid (C 14) esters (about 1%), caprylic acid (C8) esters (less than 1%), and capric acid (ClO) esters (less than 1%)

Stearic acid (C 18) is the predominant fatty acid component of the glycerides and polyethylene glycol esters in GELUCIRE® 53/10. GELUCIRE® 53/10 is known as PEG-32 glyceryl stearate (Gattefosse).

In one embodiment, the polyethylene oxide-containing fatty acid ester is a lauric acid ester, a palmitic acid ester, or a stearic acid ester (i.e., mono- and di-lauric acid esters of polyethylene glycol, mono- and di-palmitic acid esters of polyethylene glycol, mono- and di-stearic acid esters of polyethylene glycol). Mixtures of these esters can also be used.

For embodiments that include polyethylene oxide-containing fatty acid esters, the ratio of the fatty acid glycerol esters to polyethylene oxide-containing fatty acid esters is from about 20:80 to about 80:20 v/v. In one embodiment, the ratio of the fatty acid glycerol esters to polyethylene oxide-containing fatty acid esters is about 30:70 v/v. In one embodiment, the ratio of the fatty acid glycerol esters to polyethylene oxide-containing fatty acid esters is about 40:60 v/v. In one embodiment, the ratio of the fatty acid glycerol esters to polyethylene oxide-containing fatty acid esters is about 50:50 v/v. In one embodiment, the ratio of the fatty acid glycerol esters to polyethylene oxide-containing fatty acid esters is about 60:40 v/v. In one embodiment, the ratio of the fatty acid glycerol esters to polyethylene oxide-containing fatty acid esters is about 70:30 v/v.

In one embodiment, the amphotericin B formulations of the invention comprise:

(a) amphotericin B;

(b) oleic acid glycerol esters; and

(c) lauric acid esters of polyethylene glycol.

In another embodiment, the amphotericin B formulations of the invention comprise:

(a) amphotericin B;

(b) oleic acid glycerol esters; and

(c) palmitic and stearic acid esters of polyethylene glycol.

In a further embodiment, the amphotericin B formulations of the invention comprise:

(a) amphotericin B;

(b) oleic acid glycerol esters; and

(c) stearic acid esters of polyethylene glycol.

In one embodiment, the amphotericin B formulations of the invention comprise:

(a) amphotericin B;

(b) oleic acid glycerol esters;

(c) lauric acid esters of polyethylene glycol; and

(d) optionally a tocopherol polyethylene glycol succinate.

In another embodiment, the amphotericin B formulations of the invention comprise:

(a) amphotericin B;

(b) oleic acid glycerol esters;

(c) palmitic and stearic acid esters of polyethylene glycol; and

(d) optionally a tocopherol polyethylene glycol succinate.

In a further embodiment, the amphotericin B formulations of the invention comprise:

(a) amphotericin B;

(b) oleic acid glycerol esters;

(c) stearic acid esters of polyethylene glycol; and

(d) optionally a tocopherol polyethylene glycol succinate.

In one embodiment, the amphotericin B formulations of the invention include amphotericin B, PECEOL®, and GELUCIRE® 44/14. In another embodiment, the amphotericin B formulation of the invention includes amphotericin B, PECEOL®, and GELUCIRE® 50/13. In a further embodiment, the amphotericin B formulation of the invention includes amphotericin B, PECEOL®, and GELUCIRE® 53/10. In these embodiments, the ratio of PECEOL® to GELUCIRE® can be from 20:80 to 80:20 (e.g., 20:80, 30:70; 40:60; 50:50; 60:40; 70:30; and 80:20).

The preparation and characterization of representative amphotericin B formulations of the invention that include polyethylene oxide-containing fatty acid esters is described in PCT Publication No. WO 2008/144888. In certain embodiments, the present invention utilizes an amphotericin B formulations described in PCT Publication No. WO 2008/144888.

In certain embodiment, the amphotericin B formulations that include polyethylene oxide-containing fatty acid esters include amphotericin B that is both partially solubilized (dissolved) and present as solid particles to provide a fine solid dispersion. Dispersion of the formulations in aqueous media provides a nano-/microemulsion.

As noted herein, certain amphotericin B formulations optionally include a tocopherol polyethylene glycol succinate (e.g., TPGS or vitamin E TPGS). The tocopherol polyethylene glycol is included in the formulation to enhance the thermal stability of the formulation, which in turn, can increase the formulation's shelf-life, which is particularly important in tropical regions of the world where prolonged exposure to high temperatures are common and refrigerated medicinal storage is rare. For formulations in which enhanced thermal stability is desired, the formulation includes a tocopherol polyethylene glycol succinate.

Structurally, tocopherol polyethylene glycol succinates have a polyethylene glycol (PEG) covalently coupled to tocopherol (e.g., a-tocopherol or vitamin E) through a succinate linker. Because PEG is a polymer, a variety of polymer molecular weights can be used to prepare the TPGS. In one embodiment, the TPGS is tocopherol polyethylene glycol succinate 1000, in which the average molecular weight of the PEG is 1000. One suitable tocopherol polyethylene glycol succinate is vitamin E TPGS commercially available from Eastman.

As used herein, “vitamin E” refers to a family of compounds that includes α-, β-, γ-, and δ-tocopherols and the corresponding tocotrienols.

The preparation of representative amphotericin B formulations of the invention that include fatty acid glycerol esters, polyethylene oxide-containing fatty acid esters, and a tocopherol polyethylene glycol succinate is described in PCT Publication No. WO2011/050457. In certain embodiments, the present invention utilizes an amphotericin B formulation described in WO2011/050457.

In one aspect, the invention provides oral formulations of amphotericin B that are stable at the temperatures of WHO Climatic Zones 3 and 4 (30-43° C.). Four representative AmpB formulations were prepared comprising mono- and di-glycerides (Peceol), pegylated esters (Gelucire 44/14), and optionally a vitamin E-TPGS (TPGS). The compositions of the four AmpB formulations are summarized in Table 1.

TABLE 1
Compositions of Representative
AmpB Formulations

| Formu- | AmpB | Peceol/Gelucire | TPGS |
|---|---|---|---|
| lation | (mg/mL) | 44/14 (v/v) | (v/v) |
| A | 5 | 50:50 | 5 |
| B | 5 | 60:40 | 5 |
| C | 5 | 50:50 | 0 |
| D | 5 | 60:40 | 0 |

In summary, in certain aspects, the present invention provides amphotericin B formulations that can be orally administered. The amphotericin B formulations of the invention provide excellent drug solubilization, drug stability in simulated gastric and intestinal fluids, and have significant antifungal activity without the dose-limiting renal toxicity for which the parenteral formulations of amphotericin B are well known.

Protease Inhibitor Formulations

In one aspect, the present invention provides protease inhibitor formulations, methods for making the formulations, methods for administering protease inhibitors using the formulations, and methods for treating diseases treatable by protease inhibitors by administering the formulations.

In one aspect, the present invention provides protease inhibitor formulations that comprise:

(a) a protease inhibitor;

(b) one or more fatty acid glycerol esters; and

(c) one or more polyethylene oxide-containing phospholipids or one or more polyethylene oxide-containing fatty acid esters.

In another aspect, the present invention provides protease inhibitor formulations that comprise:

(a) a protease inhibitor;

(b) one or more fatty acid glycerol esters;

(c) one or more one or more polyethylene oxide-containing phospholipids or one or more polyethylene oxide-containing fatty acid esters; and

(d) optionally a tocopherol polyethylene glycol succinate.

In representative formulations of any of the formulations described herein, a protease inhibitor or pharmaceutically acceptable salt thereof is present in an amount from about 0.5 to about 10 mg/mL, about 0.1 to about 1000 mg/mL, about 0.1 to about 100 mg/mL, about 0.5 to about 50 mg/mL, or about 0.5 to about 20 mg/mL of the formulation. In one embodiment, a protease inhibitor or pharmaceutically acceptable salt thereof is present in the formulation in about 5 mg/mL or about 10 mg/mL or about 1 mg/mL or about 20 mg/mL. In one embodiment, a protease inhibitor or pharmaceutically acceptable salt thereof is present in the formulation in about 7 mg/mL. In one embodiment, a protease inhibitor or pharmaceutically acceptable salt thereof is present in the formulation in about 5 mg/mL.

In certain embodiments, the formulations include one or more fatty acid glycerol esters, and typically, a mixture of fatty acid glycerol esters. As used herein the term “fatty acid glycerol esters” refers to esters formed between glycerol and one or more fatty acids including mono-, di-, and tri-esters (i.e., glycerides). Suitable fatty acids include saturated and unsaturated fatty acids having from eight (8) to twenty-two (22) carbons atoms (i.e., C8-C22 fatty acids). In certain embodiments, suitable fatty acids include C12-C18 fatty acids.

The fatty acid glycerol esters useful in the formulations can be provided by commercially available sources. A representative source for the fatty acid glycerol esters is a mixture of mono-, di-, and triesters commercially available as PECEOL® (Gattefosse, Saint Priest Cedex, France), commonly referred to as “glyceryl oleate” or “glyceryl monooleate.” When PECEOL® is used as the source of fatty acid glycerol esters in the formulations, the fatty acid glycerol esters comprise from about 32 to about 52% by weight fatty acid monoglycerides, from about 30 to about 50% by weight fatty acid diglycerides, and from about 5 to about 20% by weight fatty acid triglycerides. The fatty acid glycerol esters comprise greater than about 60% by weight oleic acid (C18:1) mono-, di-, and triglycerides. Other fatty acid glycerol esters include esters of palmitic acid (C16) (less than about 12%), stearic acid (C18) (less than about 6%), linoleic acid (C18:2) (less than about 35%), linolenic acid (C18:3) (less than about 2%), arachidic acid (C20) (less than about 2%), and eicosenoic acid (C20:1) (less than about 2%). PECEOL® can also include free glycerol (typically about 1%). In one embodiment, the fatty acid glycerol esters comprise about 44% by weight fatty acid monoglycerides, about 45% by weight fatty acid diglycerides, and about 9% by weight fatty acid triglycerides, and the fatty acid glycerol esters comprise about 78% by weight oleic acid (C18:1) mono-, di-, and triglycerides. Other fatty acid glycerol esters include esters of palmitic acid (C16) (about 4%), stearic acid (Cl 8) (about 2%), linoleic acid (Cl 8:2) (about 12%), linolenic acid (C18:3) (less than 1%), arachidic acid (C20) (less than 1%), and eicosenoic acid (C20:1) (less than 1%).

In certain embodiments, the formulations of the invention can include glycerol in an amount less than about 10% by weight.

In certain embodiments, the formulations include a tocopherol polyethylene glycol succinate.

Formulations of the present invention offer advantages over previous formulations, including ease of oral delivery, increased stability of the protease inhibitor, increased delivery of the protease inhibitor across the blood brain barrier to the brain, and increased delivery to the lymphatic system.

In particular embodiments, the formulations of the present invention comprise one or more protease inhibitors. In particular embodiments, one or more of the protease inhibitor(s) is amprenavir, ritonavir, saquinavir, tipranavir, atazanavir, fosamprenavir, lopinavir, indinavir, darunavir, brecanavir, or nelfinavir, or a pharmaceutically acceptable salt thereof. In certain embodiments, the protease inhibitor is a compound having low solubility. In particular embodiments, the protease inhibitors have the following structures or a pharmaceutically acceptable salt thereof:

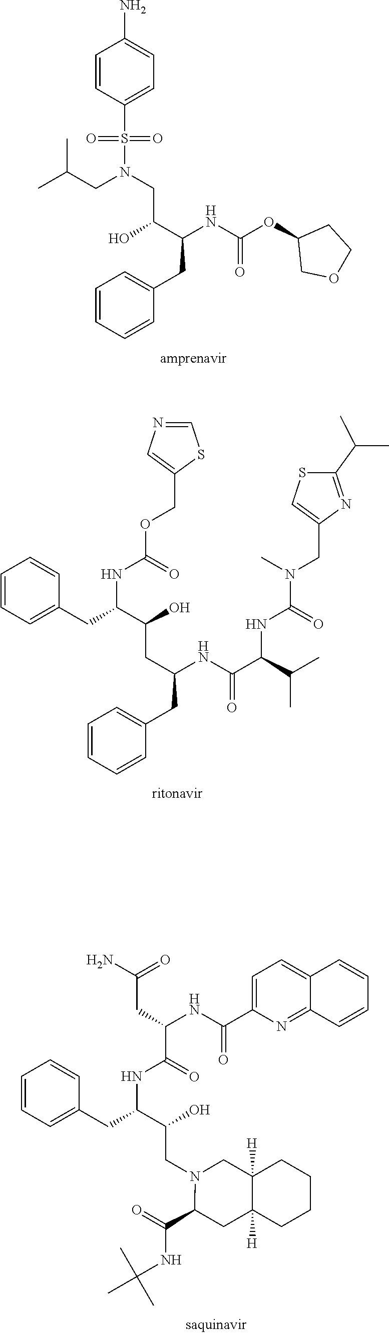 [SMILES: CC(C)CN(C[C@@H](O)[C@H](Cc1ccccc1)NC(=O)O[C@H]1CCOC1)S(=O)(=O)c1ccc(N)cc1 ; CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1 ; CC(C)(C)NC(=O)[C@@H]1C[C@@H]2CCCC[C@@H]2CN1C[C@@H](O)[C@H](Cc1ccccc1)NC(=O)[C@H](CC(N)=O)NC(=O)c1ccc2ccccc2n1]

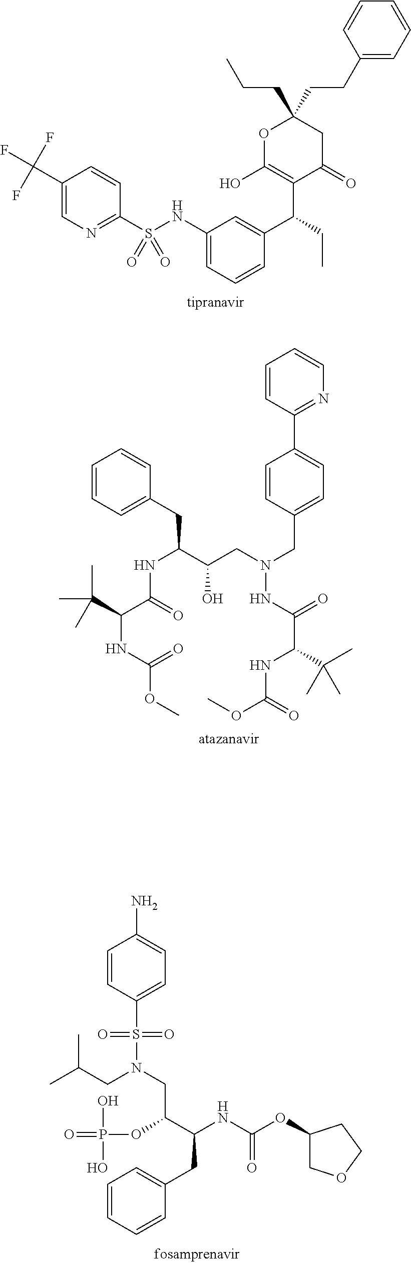

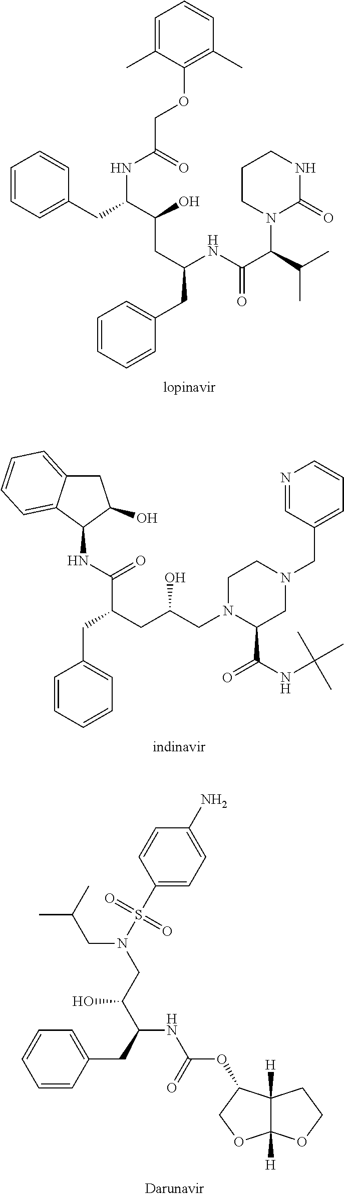 [SMILES: Cc1cccc(C)c1OCC(=O)N[C@@H](Cc1ccccc1)[C@@H](O)C[C@H](Cc1ccccc1)NC(=O)[C@H](C(C)C)N1CCCNC1=O ; CC(C)(C)NC(=O)[C@@H]1CN(Cc2cccnc2)CCN1C[C@@H](O)C[C@H](Cc1ccccc1)C(=O)N[C@H]1c2ccccc2C[C@H]1O ; CC(C)CN(C[C@@H](O)[C@H](Cc1ccccc1)NC(=O)O[C@H]1CO[C@H]2OCC[C@H]21)S(=O)(=O)c1ccc(N)cc1]

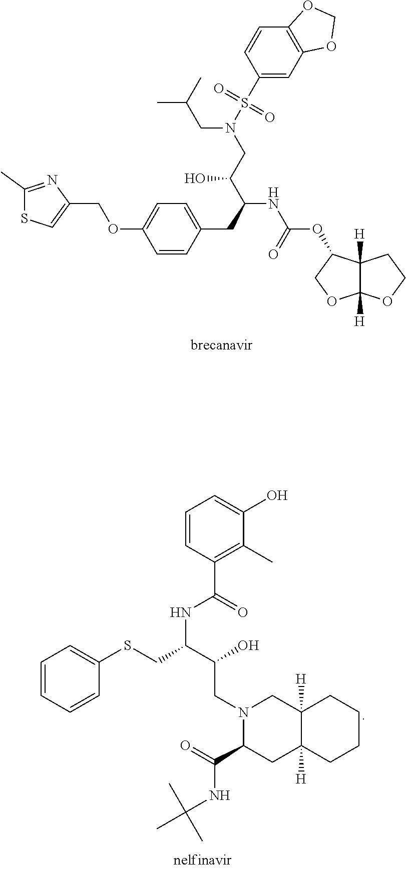 [SMILES: Cc1nc(COc2ccc(C[C@H](NC(=O)O[C@H]3CO[C@H]4OCC[C@H]43)[C@H](O)CN(CC(C)C)S(=O)(=O)c3ccc4c(c3)OCO4)cc2)cs1 ; Cc1c(O)cccc1C(=O)N[C@@H](CSc1ccccc1)[C@H](O)CN1C[C@H]2CCCC[C@H]2C[C@H]1C(=O)NC(C)(C)C]

In certain embodiments, a protease inhibitor formulation of the present invention is provided to a subject in need thereof as part of a highly active antiretroviral therapy (HAART). HAART is an aggressive treatment regimen used to suppress HIV viral replication and progression of HIV. The usual HAART regimen combines three or more different drugs such as two nucleoside reverse transcriptase inhibitors (NRTIs) and a protease inhibitor (PI); two NRTIs and a non-nucleoside reverse transcriptase inhibitor (NNRTI); one NRTI, one PI and one NNRTI; or other such combination. In certain embodiments, it includes efavirenz, teofovir, and emtricitabine; rilpivirine, tenofovir, and emtricitabine; elvitegravir, cobicistat, tenofovir, and emtricitabine; dolutegravir, abacavir, and lamivudine; or tenofovir disoproxil fumarate (TDF)/emtricitabine (FTC), efavirenz, atazanavir/ritonavir, and darunavir. In particular embodiments, formulations of the present invention comprise two or more portease inhibitors, while in other embodiments, formulations of the present invention comprise active agents constituting any of the HAART regimens.

Protease Inhibitor Formulations: Polyethylene Oxide-Containing Phospholipids (DSPE-PEGs)

In certain embodiments, the protease inhibitor formulations include one or more polyethoxylated lipids. In one embodiment, the polyethoxylated lipids are polyethylene oxide-containing phospholipids, or a mixture of polyethylene oxide-containing phospholipids. In another embodiment, the polyethoxylated lipids are polyethylene oxide-containing fatty acid esters, or a mixture of polyethylene oxide-containing fatty acid esters.

Accordingly, in one embodiment, the protease inhibitor formulations of the invention include:

(a) a protease inhibitor;

(b) one or more fatty acid glycerol esters; and

(c) one or more polyethylene oxide-containing phospholipids.

As used herein, the term “polyethylene oxide-containing phospholipid” refers to a phospholipid that includes a polyethylene oxide group (i.e., polyethylene glycol group) covalently coupled to the phospholipid, typically through a carbamate or an ester bond. Phospholipids are derived from glycerol and can include a phosphate ester group and two fatty acid ester groups. Suitable fatty acids include saturated and unsaturated fatty acids having from eight (8) to twenty-two (22) carbons atoms (i.e., C8-C22 fatty acids). In certain embodiments, suitable fatty acids include saturated C 12-Cl 8 fatty acids. Representative polyethylene oxide-containing phospholipids include C8-C22 saturated fatty acid esters of a phosphatidyl ethanolamine polyethylene glycol salt. In certain embodiments, suitable fatty acids include saturated C 12-Cl 8 fatty acids.

The molecular weight of the polyethylene oxide group of the polyethylene oxide-containing phospholipid can be varied to optimize the solubility of the therapeutic agent (e.g., protease inhibitor) in the formulation. Representative average molecular weights for the polyethylene oxide groups can be from about 200 to about 5000 (e.g., PEG 200 to PEG 5000).

In one embodiment, the polyethylene oxide-containing phospholipids are distearoyl phosphatidyl ethanolamine polyethylene glycol salts. Representative distearoylphosphatidyl ethanolamine polyethylene glycol salts include distearoylphosphatidyl ethanolamine polyethylene glycol 350 (DSPE-PEG-350) salts, distearoylphosphatidyl ethanolamine polyethylene glycol 550 (DSPE-PEG-550) salts, distearoylphosphatidyl ethanolamine polyethylene glycol 750 (DSPE-PEG-750) salts, distearoylphosphatidyl ethanolamine polyethylene glycol 1000 (DSPE-PEG-1000) salts, distearoylphosphatidyl ethanolamine polyethylene glycol 1500 (DSPE-PEG-1500) salts, and distearoylphosphatidyl ethanolamine polyethylene glycol 2000 (DSPE-PEG-2000) salts. Mixtures can also be used. For the distearoylphosphatidyl ethanolamine polyethylene glycol salts above, the number (e.g., 350, 550, 750, 1000, and 2000) designates the average molecular weight of the polyethylene oxide group. The abbreviations for the salts used herein is provided in parentheses above.

Suitable distearoylphosphatidyl ethanolamine polyethylene glycol salts include ammonium and sodium salts. The chemical structure of distearoylphosphatidyl ethanolamine polyethylene glycol 2000 (DSPE-PEG-2000) ammonium salt is illustrated in FIG. 1B. Referring to FIG. 1B, the polyethylene oxide-containing phospholipid includes a phosphate ester group and two fatty acid ester (stearate) groups, and a polyethylene oxide group covalently coupled to the amino group of the phosphatidyl ethanolamine through a carbamate bond.

As noted above, the polyethylene oxide-containing phospholipid affects the ability of the formulation to solubilize a therapeutic agent. In general, the greater the amount of polyethylene oxide-containing phospholipid, the greater the solubilizing capacity of the formulation for difficultly soluble therapeutic agents. The polyethylene oxide-containing phospholipid can be present in the formulation in an amount from about 1 mM to about 30 mM based on the volume of the formulation. In certain embodiments, the distearoylphosphatidyl ethanolamine polyethylene glycol salt is present in the formulation in an amount from 1 mM to about 30 mM based on the volume of the formulation. In one embodiment, the distearoylphosphatidyl ethanolamine polyethylene glycol salt is present in the formulation in about 15 mM based on the volume of the formulation.

In one embodiment, the protease inhibitor formulations of the invention comprise:

(a) a protease inhibitor;

(b) oleic acid glycerol esters; and

(c) a distearoylphosphatidyl ethanolamine polyethylene glycol salt.

In one embodiment, the protease inhibitor formulation of the invention includes amphotericin B, PECEOL®, and a distearoylphosphatidyl ethanolamine polyethylene glycol salt. In this embodiment, the distearoylphosphatidyl ethanolamine polyethylene glycol salt is present in an amount up to about 30 mM. The preparation of representative protease inhibitor formulations of the invention that include polyethylene oxide-containing phospholipids is based on a modification of the procedure described in PCT Publication No. WO2008/144888 for AmpB formulations.

In certain embodiments, the protease inhibitor formulations that include polyethylene oxide-containing phospholipids include amphotericin B that is both partially solubilized (dissolved) and present as solid particles to provide a fine solid dispersion. Dispersion of the formulation in aqueous media provides a nano-/microemulsion having emulsion droplets that range in size from about 50 nm to about 5 μm.

Protease Inhibitor Formulations: Polyethylene Oxide-Containing Fatty Acid Esters

In certain embodiments, the protease inhibitor formulations include one or more polyethoxylated lipids such as polyethylene oxide-containing phospholipids or one or more polyethylene oxide-containing fatty acid esters, and typically, a mixture of polyethylene oxide-containing phospholipids or a mixture of polyethylene oxide-containing fatty acid esters.

Accordingly, in one embodiment, the protease inhibitor formulations of the invention comprise:

(a) a protease inhibitor;

(b) one or more fatty acid glycerol esters; and

(c) one or more polyethylene oxide-containing fatty acid esters.

In another embodiment, the protease inhibitor formulations of the invention comprise:

(a) a protease inhibitor;

(b) one or more fatty acid glycerol esters;

(c) one or more polyethylene oxide-containing fatty acid esters; and

(d) optionally a tocopherol polyethylene glycol succinate.

As used herein, the term “polyethylene oxide-containing fatty acid ester” refers to a fatty acid ester that includes a polyethylene oxide group (i.e., polyethylene glycol group) covalently coupled to the fatty acid through an ester bond. Polyethylene oxide-containing fatty acid esters include mono- and di-fatty acid esters of polyethylene glycol. Suitable polyethylene oxide-containing fatty acid esters are derived from fatty acids including saturated and unsaturated fatty acids having from eight (8) to twenty-two (22) carbons atoms (i.e., a polyethylene oxide ester of a C8-C22 fatty acid). In certain embodiments, suitable polyethylene oxide-containing fatty acid esters are derived from fatty acids including saturated and unsaturated fatty acids having from twelve (12) to eighteen (18) carbons atoms (i.e., a polyethylene oxide ester of a C 12-Cl 8 fatty acid). Representative polyethylene oxide-containing fatty acid esters include saturated C8-C22 fatty acid esters. In certain embodiments, suitable polyethylene oxide-containing fatty acid esters include saturated C 12-Cl 8 fatty acids.

The molecular weight of the polyethylene oxide group of the polyethylene oxide-containing fatty acid ester can be varied to optimize the solubility of the therapeutic agent (e.g., protease inhibitor) in the formulation. Representative average molecular weights for the polyethylene oxide groups can be from about 350 to about 2000. In one embodiment, the average molecular weight for the polyethylene oxide group is about 1500.

In this embodiment, the protease inhibitor formulations include one or more polyethylene oxide-containing fatty acid esters, and typically, a mixture of polyethylene oxide-containing fatty acid esters (mono- and di-fatty acid esters of polyethylene glycol). The polyethylene oxide-containing fatty acid esters useful in the formulations can be provided by commercially available sources. Representative polyethylene oxide-containing fatty acid esters (mixtures of mono- and diesters) are commercially available under the designation GELUCIRE® (Gattefosse, Saint Priest Cedex, France). Suitable polyethylene oxide-containing fatty acid esters can be provided by GELUCIRE® 44/14, GELUCIRE® 50/13, and GELUCIRE® 53/10. The numerals in these designations refer to the melting point and hydrophilic/lipophilic balance (HLB) of these materials, respectively. GELUCIRE® 44/14, GELUCIRE® 50/13, and GELUCIRE® 53/10 are mixtures of (a) mono-, di-, and triesters of glycerol (glycerides) and (b) mono- and diesters of polyethylene glycol (macrogols). The GELUCIRES can also include free polyethylene glycol (e.g., PEG 1500).

Laurie acid (C 12) is the predominant fatty acid component of the glycerides and polyethylene glycol esters in GELUCIRE® 44/14. GELUCIRE® 44/14 is referred to as a mixture of glyceryl dilaurate (lauric acid diester with glycerol) and PEG dilaurate (lauric acid diester with polyethylene glycol), and is commonly known as PEG-32 glyceryl laurate (Gattefosse) lauroyl macrogol-32 glycerides EP, or lauroyl polyoxylglycerides USP/NF. GELUCIRE® 44/14 is produced by the reaction of hydrogenated palm kernel oil with polyethylene glycol (average molecular weight 1500). GELUCIRE® 44/14 includes about 20% mono-, di- and, triglycerides, about 72% mono- and di-fatty acid esters of polyethylene glycol 1500, and about 8% polyethylene glycol 1500.

GELUCIRE® 44/14 includes lauric acid (C 12) esters (30 to 50%), myristic acid (C14) esters (5 to 25%), palmitic acid (C16) esters (4 to 25%), stearic acid (C18) esters (5 to 35%), caprylic acid (C8) esters (less than 15%), and capric acid (ClO) esters (less than 12%). GELUCIRE® 44/14 may also include free glycerol (typically less than about 1%). In a representative formulation, GELUCIRE® 44/14 includes lauric acid (C12) esters (about 47%), myristic acid (C14) esters (about 18%), palmitic acid (C16) esters (about 10%), stearic acid (C18) esters (about 11%), caprylic acid (C8) esters (about 8%), and capric acid (ClO) esters (about 12%).

Palmitic acid (C16) (40-50%) and stearic acid (C18) (48-58%) are the predominant fatty acid components of the glycerides and polyethylene glycol esters in GELUCIRE® 50/13. GELUCIRE® 50/13 is known as PEG-32 glyceryl palmitostearate (Gattefosse), stearoyl macrogolglycerides EP, or stearoyl polyoxylglycerides USP/NF). GELUCIRE® 50/13 includes palmitic acid (Cl 6) esters (40 to 50%), stearic acid (C 18) esters (48 to 58%) (stearic and palmitic acid esters greater than about 90%), lauric acid (C12) esters (less than 5%), myristic acid (C14) esters (less than 5%), caprylic acid (C8) esters (less than 3%), and capric acid (ClO) esters (less than 3%). GELUCIRE® 50/13 may also include free glycerol (typically less than about 1%). In a representative formulation, GELUCIRE® 50/13 includes palmitic acid (C16) esters (about 43%), stearic acid (C 18) esters (about 54%) (stearic and palmitic acid esters about 97%), lauric acid (C 12) esters (less than 1%), myristic acid (C 14) esters (about 1%), caprylic acid (C8) esters (less than 1%), and capric acid (ClO) esters (less than 1%)

Stearic acid (C 18) is the predominant fatty acid component of the glycerides and polyethylene glycol esters in GELUCIRE® 53/10. GELUCIRE® 53/10 is known as PEG-32 glyceryl stearate (Gattefosse).

In one embodiment, the polyethylene oxide-containing fatty acid ester is a lauric acid ester, a palmitic acid ester, or a stearic acid ester (i.e., mono- and di-lauric acid esters of polyethylene glycol, mono- and di-palmitic acid esters of polyethylene glycol, mono- and di-stearic acid esters of polyethylene glycol). Mixtures of these esters can also be used.

For embodiments that include polyethylene oxide-containing fatty acid esters, the ratio of the fatty acid glycerol esters to polyethylene oxide-containing fatty acid esters is from about 20:80 to about 80:20 v/v. In one embodiment, the ratio of the fatty acid glycerol esters to polyethylene oxide-containing fatty acid esters is about 30:70 v/v. In one embodiment, the ratio of the fatty acid glycerol esters to polyethylene oxide-containing fatty acid esters is about 40:60 v/v. In one embodiment, the ratio of the fatty acid glycerol esters to polyethylene oxide-containing fatty acid esters is about 50:50 v/v. In one embodiment, the ratio of the fatty acid glycerol esters to polyethylene oxide-containing fatty acid esters is about 60:40 v/v. In one embodiment, the ratio of the fatty acid glycerol esters to polyethylene oxide-containing fatty acid esters is about 70:30 v/v.

In one embodiment, the protease inhibitor formulations of the invention comprise:

(a) a protease inhibitor;

(b) oleic acid glycerol esters; and

(c) lauric acid esters of polyethylene glycol.

In another embodiment, the protease inhibitor formulations of the invention comprise:

(a) a protease inhibitor;

(b) oleic acid glycerol esters; and

(c) palmitic and stearic acid esters of polyethylene glycol.

In a further embodiment, the protease inhibitor formulations of the invention comprise:

(a) a protease inhibitor;

(b) oleic acid glycerol esters; and

(c) stearic acid esters of polyethylene glycol.

In one embodiment, the protease inhibitor formulations of the invention comprise:

(a) a protease inhibitor;

(b) oleic acid glycerol esters;

(c) lauric acid esters of polyethylene glycol; and

(d) optionally a tocopherol polyethylene glycol succinate.

In another embodiment, the protease inhibitor formulations of the invention comprise:

(a) a protease inhibitor;

(b) oleic acid glycerol esters;

(c) palmitic and stearic acid esters of polyethylene glycol; and

(d) optionally a tocopherol polyethylene glycol succinate.

In a further embodiment, the protease inhibitor formulations of the invention comprise:

(a) a protease inhibitor;

(b) oleic acid glycerol esters;

(c) stearic acid esters of polyethylene glycol; and

(d) optionally a tocopherol polyethylene glycol succinate.

In one embodiment, the protease inhibitor formulation of the invention includes a protease inhibitor, PECEOL®, and GELUCIRE® 44/14. In another embodiment, the protease inhibitor formulation of the invention includes a protease inhibitor, PECEOL®, and GELUCIRE® 50/13. In a further embodiment, the protease inhibitor formulation of the invention includes a protease inhibitor, PECEOL®, and GELUCIRE® 53/10. In these embodiments, the ratio of PECEOL® to GELUCIRE® can be from 20:80 to 80:20 (e.g., 20:80, 30:70; 40:60; 50:50; 60:40; 70:30; and 80:20).

The preparation and characterization of representative protease inhibitor formulations of the invention that include polyethylene oxide-containing fatty acid esters is based on a modification of the procedure described in PCT Publication No. WO 2008/144888 for AmpB formulation.

In certain embodiment, the protease inhibitor formulations that include polyethylene oxide-containing fatty acid esters include protease inhibitor that is both partially solubilized (dissolved) and present as solid particles to provide a fine solid dispersion. Dispersion of the formulations in aqueous media provides a nano-/microemulsion.

As noted herein, certain protease inhibitor formulations optionally include a tocopherol polyethylene glycol succinate (e.g., TPGS or vitamin E TPGS). The tocopherol polyethylene glycol is included in the formulation to enhance the thermal stability of the formulation, which in turn, can increase the formulation's shelf-life, which is particularly important in tropical regions of the world where prolonged exposure to high temperatures are common and refrigerated medicinal storage is rare. For formulations in which enhanced thermal stability is desired, the formulation includes a tocopherol polyethylene glycol succinate.

Structurally, tocopherol polyethylene glycol succinates have a polyethylene glycol (PEG) covalently coupled to tocopherol (e.g., a-tocopherol or vitamin E) through a succinate linker. Because PEG is a polymer, a variety of polymer molecular weights can be used to prepare the TPGS. In one embodiment, the TPGS is tocopherol polyethylene glycol succinate 1000, in which the average molecular weight of the PEG is 1000. One suitable tocopherol polyethylene glycol succinate is vitamin E TPGS commercially available from Eastman.

As used herein, “vitamin E” refers to a family of compounds that includes α-, β-, γ-, and δ-tocopherols and the corresponding tocotrienols.

The preparation of representative protease inhibitor formulations of the invention that include fatty acid glycerol esters, polyethylene oxide-containing fatty acid esters, and a tocopherol polyethylene glycol succinate is based on the procedure described in PCT Publication No. WO2011/050457 for AmpB formulations.

In one aspect, the invention provides oral formulations of protease inhibitor that are stable at the temperatures of WHO Climatic Zones 3 and 4 (30-43° C.). Four representative protease inhibitor formulations comprising mono- and di-glycerides (Peceol), pegylated esters (Gelucire 44/14), and optionally a vitamin E-TPGS (TPGS) are summarized in Table 2.

TABLE 2
Compositions of Representative
Protease Inhibitor Formulations

| Formu- | Protease | Peceol/Gelucire | TPGS |
|---|---|---|---|
| lation | Inhibitor | 44/14 (v/v) | (v/v) |
| A | variable | 50:50 | 5 |
| B | variable | 60:40 | 5 |
| C | variable | 50:50 | 0 |
| D | variable | 60:40 | 0 |

In summary, in certain aspects, the present invention provides protease inhibitor formulations that can be orally administered. The protease inhibitor formulations of the invention provide excellent drug solubilization, drug stability in simulated gastric and intestinal fluids, and have significant activity.

### AmbB and Protease Inhibitor Formulations: Polyethylene Oxide-Containing Phospholipids (DSPE-PEGs)

In certain embodiment, the present invention includes formulations comprising both AmpB and one or more protease inhibitors. In certain embodiment, the formulations include one or more polyethoxylated lipids. In one embodiment, the polyethoxylated lipids are polyethylene oxide-containing phospholipids, or a mixture of polyethylene oxide-containing phospholipids. In another embodiment, the polyethoxylated lipids are polyethylene oxide-containing fatty acid esters, or a mixture of polyethylene oxide-containing fatty acid esters. In representative formulations of any of the formulations described herein, amphotericin B is present in an amount from about 0.5 to about 10 mg/mL, about 0.1 to about 1000 mg/mL, about 0.1 to about 100 mg/mL, about 0.5 to about 50 mg/mL, or about 0.5 to about 20 mg/mL of the formulation. In one embodiment, amphotericin B or pharmaceutically acceptable salt thereof is present in the formulation in about 5 mg/mL or about 10 mg/mL or about 1 mg/mL or about 20 mg/mL. In one embodiment, amphotericin B or its pharmaceutically acceptable salt thereof is present in the formulation in about 7 mg/mL. In representative formulations of any of the formulations described herein, a protease inhibitor or a pharmaceutically acceptable salt thereof is present in an amount from about 0.5 to about 10 mg/mL, about 0.1 to about 1000 mg/mL, about 0.1 to about 100 mg/mL, about 0.5 to about 50 mg/mL, or about 0.5 to about 20 mg/mL of the formulation. In one embodiment, a protease inhibitor or a pharmaceutically acceptable salt thereof is present in the formulation in about 5 mg/mL or about 10 mg/mL or about 1 mg/mL or about 20 mg/mL. In one embodiment, a protease inhibitor or a pharmaceutically acceptable salt thereof is present in the formulation in about 7 mg/mL.

In particular embodiments, the formulations comprise two or more protease inhibitors. In particular embodiments, the protease inhibitor is any of those described herein, which may be provided to the subject in any of the amounts described therein.

Accordingly, in one embodiment, the AmpB and protease inhibitor formulations of the invention comprise:

(a) AmpB;

(b) a protease inhibitor;

(c) one or more fatty acid glycerol esters; and

(d) one or more polyethylene oxide-containing phospholipids.

As used herein, the term “polyethylene oxide-containing phospholipid” refers to a phospholipid that includes a polyethylene oxide group (i.e., polyethylene glycol group) covalently coupled to the phospholipid, typically through a carbamate or an ester bond. Phospholipids are derived from glycerol and can include a phosphate ester group and two fatty acid ester groups. Suitable fatty acids include saturated and unsaturated fatty acids having from eight (8) to twenty-two (22) carbons atoms (i.e., C8-C22 fatty acids). In certain embodiments, suitable fatty acids include saturated C 12-Cl 8 fatty acids. Representative polyethylene oxide-containing phospholipids include C8-C22 saturated fatty acid esters of a phosphatidyl ethanolamine polyethylene glycol salt. In certain embodiments, suitable fatty acids include saturated C 12-Cl 8 fatty acids.

The molecular weight of the polyethylene oxide group of the polyethylene oxide-containing phospholipid can be varied to optimize the solubility of the therapeutic agent (e.g., protease inhibitor) in the formulation. Representative average molecular weights for the polyethylene oxide groups can be from about 200 to about 5000 (e.g., PEG 200 to PEG 5000).

In one embodiment, the polyethylene oxide-containing phospholipids are distearoyl phosphatidyl ethanolamine polyethylene glycol salts. Representative distearoylphosphatidyl ethanolamine polyethylene glycol salts include distearoylphosphatidyl ethanolamine polyethylene glycol 350 (DSPE-PEG-350) salts, distearoylphosphatidyl ethanolamine polyethylene glycol 550 (DSPE-PEG-550) salts, distearoylphosphatidyl ethanolamine polyethylene glycol 750 (DSPE-PEG-750) salts, distearoylphosphatidyl ethanolamine polyethylene glycol 1000 (DSPE-PEG-1000) salts, distearoylphosphatidyl ethanolamine polyethylene glycol 1500 (DSPE-PEG-1500) salts, and distearoylphosphatidyl ethanolamine polyethylene glycol 2000 (DSPE-PEG-2000) salts. Mixtures can also be used. For the distearoylphosphatidyl ethanolamine polyethylene glycol salts above, the number (e.g., 350, 550, 750, 1000, and 2000) designates the average molecular weight of the polyethylene oxide group. The abbreviations for these salts used herein is provided in parentheses above.

Suitable distearoylphosphatidyl ethanolamine polyethylene glycol salts include ammonium and sodium salts. The chemical structure of distearoylphosphatidyl ethanolamine polyethylene glycol 2000 (DSPE-PEG-2000) ammonium salt is illustrated in FIG. 1B. Referring to FIG. 1B, the polyethylene oxide-containing phospholipid includes a phosphate ester group and two fatty acid ester (stearate) groups, and a polyethylene oxide group covalently coupled to the amino group of the phosphatidyl ethanolamine through a carbamate bond.

As noted above, the polyethylene oxide-containing phospholipid affects the ability of the formulation to solubilize a therapeutic agent. In general, the greater the amount of polyethylene oxide-containing phospholipid, the greater the solubilizing capacity of the formulation for difficultly soluble therapeutic agents. The polyethylene oxide-containing phospholipid can be present in the formulation in an amount from about 1 mM to about 30 mM based on the volume of the formulation. In certain embodiments, the distearoylphosphatidyl ethanolamine polyethylene glycol salt is present in the formulation in an amount from 1 mM to about 30 mM based on the volume of the formulation. In one embodiment, the distearoylphosphatidyl ethanolamine polyethylene glycol salt is present in the formulation in about 15 mM based on the volume of the formulation.

In one embodiment, the AmpB and protease inhibitor formulations of the invention include:

(a) AmpB;

(b) a protease inhibitor;

(c) oleic acid glycerol esters; and

(d) a distearoylphosphatidyl ethanolamine polyethylene glycol salt.

In one embodiment, the AmpB and protease inhibitor formulation of the invention includes amphotericin B, a protease inhibitor, PECEOL®, and a distearoylphosphatidyl ethanolamine polyethylene glycol salt. In certain embodiments, the distearoylphosphatidyl ethanolamine polyethylene glycol salt is present in an amount up to about 30 mM. The preparation of representative AmpB and protease inhibitor formulations of the invention that include polyethylene oxide-containing phospholipids is based on a modification of the procedure described in PCT Publication No. WO2008/144888 for AmpB formulations.

In certain embodiments, the AmpB and protease inhibitor formulations that include polyethylene oxide-containing phospholipids include amphotericin B that is both partially solubilized (dissolved) and present as solid particles to provide a fine solid dispersion. Dispersion of the formulation in aqueous media provides a nano-/microemulsion having emulsion droplets that range in size from about 50 nm to about 5 μm.

### AmpB and Protease Inhibitor Formulations: Polyethylene Oxide-Containing Fatty Acid Esters

As noted above, certain AmpB and protease inhibitor formulations include one or more polyethoxylated lipids such as polyethylene oxide-containing phospholipids or one or more polyethylene oxide-containing fatty acid esters, and typically, a mixture of polyethylene oxide-containing phospholipids or a mixture of polyethylene oxide-containing fatty acid esters.

Accordingly, in one embodiment, the AmpB and protease inhibitor formulations of the invention comprises:

(a) AmpB;

(b) a protease inhibitor;

(c) one or more fatty acid glycerol esters; and

(d) one or more polyethylene oxide-containing fatty acid esters.

In another embodiment, the protease inhibitor formulations of the invention comprises:

(a) ampB

(b) a protease inhibitor;

(c) one or more fatty acid glycerol esters;

(d) one or more polyethylene oxide-containing fatty acid esters; and

(e) optionally a tocopherol polyethylene glycol succinate.

As used herein, the term “polyethylene oxide-containing fatty acid ester” refers to a fatty acid ester that includes a polyethylene oxide group (i.e., polyethylene glycol group) covalently coupled to the fatty acid through an ester bond. Polyethylene oxide-containing fatty acid esters include mono- and di-fatty acid esters of polyethylene glycol. Suitable polyethylene oxide-containing fatty acid esters are derived from fatty acids including saturated and unsaturated fatty acids having from eight (8) to twenty-two (22) carbons atoms (i.e., a polyethylene oxide ester of a C8-C22 fatty acid). In certain embodiments, suitable polyethylene oxide-containing fatty acid esters are derived from fatty acids including saturated and unsaturated fatty acids having from twelve (12) to eighteen (18) carbons atoms (i.e., a polyethylene oxide ester of a C 12-Cl 8 fatty acid). Representative polyethylene oxide-containing fatty acid esters include saturated C8-C22 fatty acid esters. In certain embodiments, suitable polyethylene oxide-containing fatty acid esters include saturated C 12-Cl 8 fatty acids.

The molecular weight of the polyethylene oxide group of the polyethylene oxide-containing fatty acid ester can be varied to optimize the solubility of the therapeutic agent (e.g., protease inhibitor) in the formulation. Representative average molecular weights for the polyethylene oxide groups can be from about 350 to about 2000. In one embodiment, the average molecular weight for the polyethylene oxide group is about 1500.

In this embodiment, the protease inhibitor formulations include one or more polyethylene oxide-containing fatty acid esters, and typically, a mixture of polyethylene oxide-containing fatty acid esters (mono- and di-fatty acid esters of polyethylene glycol). The polyethylene oxide-containing fatty acid esters useful in the formulations can be provided by commercially available sources. Representative polyethylene oxide-containing fatty acid esters (mixtures of mono- and diesters) are commercially available under the designation GELUCIRE® (Gattefosse, Saint Priest Cedex, France). Suitable polyethylene oxide-containing fatty acid esters can be provided by GELUCIRE® 44/14, GELUCIRE® 50/13, and GELUCIRE® 53/10. The numerals in these designations refer to the melting point and hydrophilic/lipophilic balance (HLB) of these materials, respectively. GELUCIRE® 44/14, GELUCIRE® 50/13, and GELUCIRE® 53/10 are mixtures of (a) mono-, di-, and triesters of glycerol (glycerides) and (b) mono- and diesters of polyethylene glycol (macrogols). The GELUCIRES can also include free polyethylene glycol (e.g., PEG 1500).

Laurie acid (C 12) is the predominant fatty acid component of the glycerides and polyethylene glycol esters in GELUCIRE® 44/14. GELUCIRE® 44/14 is referred to as a mixture of glyceryl dilaurate (lauric acid diester with glycerol) and PEG dilaurate (lauric acid diester with polyethylene glycol), and is commonly known as PEG-32 glyceryl laurate (Gattefosse) lauroyl macrogol-32 glycerides EP, or lauroyl polyoxylglycerides USP/NF. GELUCIRE® 44/14 is produced by the reaction of hydrogenated palm kernel oil with polyethylene glycol (average molecular weight 1500). GELUCIRE® 44/14 includes about 20% mono-, di- and, triglycerides, about 72% mono- and di-fatty acid esters of polyethylene glycol 1500, and about 8% polyethylene glycol 1500.

GELUCIRE® 44/14 includes lauric acid (C 12) esters (30 to 50%), myristic acid (C14) esters (5 to 25%), palmitic acid (C16) esters (4 to 25%), stearic acid (C18) esters (5 to 35%), caprylic acid (C8) esters (less than 15%), and capric acid (ClO) esters (less than 12%). GELUCIRE® 44/14 may also include free glycerol (typically less than about 1%). In a representative formulation, GELUCIRE® 44/14 includes lauric acid (C12) esters (about 47%), myristic acid (C14) esters (about 18%), palmitic acid (C16) esters (about 10%), stearic acid (C18) esters (about 11%), caprylic acid (C8) esters (about 8%), and capric acid (ClO) esters (about 12%).

Palmitic acid (C16) (40-50%) and stearic acid (C18) (48-58%) are the predominant fatty acid components of the glycerides and polyethylene glycol esters in GELUCIRE® 50/13. GELUCIRE® 50/13 is known as PEG-32 glyceryl palmitostearate (Gattefosse), stearoyl macrogolglycerides EP, or stearoyl polyoxylglycerides USP/NF). GELUCIRE® 50/13 includes palmitic acid (Cl 6) esters (40 to 50%), stearic acid (C 18) esters (48 to 58%) (stearic and palmitic acid esters greater than about 90%), lauric acid (C12) esters (less than 5%), myristic acid (C14) esters (less than 5%), caprylic acid (C8) esters (less than 3%), and capric acid (ClO) esters (less than 3%). GELUCIRE® 50/13 may also include free glycerol (typically less than about 1%). In a representative formulation, GELUCIRE® 50/13 includes palmitic acid (C16) esters (about 43%), stearic acid (C 18) esters (about 54%) (stearic and palmitic acid esters about 97%), lauric acid (C 12) esters (less than 1%), myristic acid (C 14) esters (about 1%), caprylic acid (C8) esters (less than 1%), and capric acid (ClO) esters (less than 1%)

Stearic acid (C 18) is the predominant fatty acid component of the glycerides and polyethylene glycol esters in GELUCIRE® 53/10. GELUCIRE® 53/10 is known as PEG-32 glyceryl stearate (Gattefosse).

In one embodiment, the polyethylene oxide-containing fatty acid ester is a lauric acid ester, a palmitic acid ester, or a stearic acid ester (i.e., mono- and di-lauric acid esters of polyethylene glycol, mono- and di-palmitic acid esters of polyethylene glycol, mono- and di-stearic acid esters of polyethylene glycol). Mixtures of these esters can also be used.

For embodiments that include polyethylene oxide-containing fatty acid esters, the ratio of the fatty acid glycerol esters to polyethylene oxide-containing fatty acid esters is from about 20:80 to about 80:20 v/v. In one embodiment, the ratio of the fatty acid glycerol esters to polyethylene oxide-containing fatty acid esters is about 30:70 v/v. In one embodiment, the ratio of the fatty acid glycerol esters to polyethylene oxide-containing fatty acid esters is about 40:60 v/v. In one embodiment, the ratio of the fatty acid glycerol esters to polyethylene oxide-containing fatty acid esters is about 50:50 v/v. In one embodiment, the ratio of the fatty acid glycerol esters to polyethylene oxide-containing fatty acid esters is about 60:40 v/v. In one embodiment, the ratio of the fatty acid glycerol esters to polyethylene oxide-containing fatty acid esters is about 70:30 v/v.

In one embodiment, the AmpB and protease inhibitor formulations of the invention comprise:

(a) AmpB;

(b) a protease inhibitor;

(c) oleic acid glycerol esters; and

(d) lauric acid esters of polyethylene glycol.

In another embodiment, the AmpB and protease inhibitor formulations of the invention comprise:

(a) AmpB;

(b) a protease inhibitor;

(c) oleic acid glycerol esters; and

(d) palmitic and stearic acid esters of polyethylene glycol.

In a further embodiment, the AmpB and protease inhibitor formulations of the invention comprise:

(a) AmpB;

(b) a protease inhibitor;

(c) oleic acid glycerol esters; and

(d) stearic acid esters of polyethylene glycol.

In one embodiment, the AmpB and protease inhibitor formulations of the invention comprise:

(a) AmpB;

(b) a protease inhibitor;

(c) oleic acid glycerol esters;

(d) lauric acid esters of polyethylene glycol; and

(e) optionally a tocopherol polyethylene glycol succinate.

In another embodiment, the AmpB and protease inhibitor formulations of the invention comprise:

(a) AmpB;

(b) a protease inhibitor;

(c) oleic acid glycerol esters;

(d) palmitic and stearic acid esters of polyethylene glycol; and

(e) optionally a tocopherol polyethylene glycol succinate.

In a further embodiment, the AmpB and protease inhibitor formulations of the invention comprise:

(a) AmpB;

(b) a protease inhibitor;

(c) oleic acid glycerol esters;

(d) stearic acid esters of polyethylene glycol; and

(e) optionally a tocopherol polyethylene glycol succinate.

In one embodiment, the ampB and protease inhibitor formulation of the invention includes amphotericin B, a protease inhibitor, PECEOL®, and GELUCIRE® 44/14. In another embodiment, the AmpB and protease inhibitor formulation of the invention includes amphotericin B, a protease inhibitor, PECEOL®, and GELUCIRE® 50/13. In a further embodiment, the AmpB and protease inhibitor formulation of the invention includes amphotericin B, a protease inhibitor, PECEOL®, and GELUCIRE® 53/10. In these embodiments, in certain embodiments, the ratio of PECEOL® to GELUCIRE® can be from 20:80 to 80:20 (e.g., 20:80, 30:70; 40:60; 50:50; 60:40; 70:30; and 80:20).

The preparation and characterization of representative AmpB and protease inhibitor formulations of the invention that include polyethylene oxide-containing fatty acid esters is based on a modification of the procedure described in PCT Publication No. WO 2008/144888 for AmpB formulation.

In certain embodiment, the AmpB and protease inhibitor formulations that include polyethylene oxide-containing fatty acid esters include AmpB and/or protease inhibitor that is both partially solubilized (dissolved) and present as solid particles to provide a fine solid dispersion. Dispersion of the formulations in aqueous media provides a nano-/microemulsion.

In certain embodiments, AmpB and protease inhibitor formulations optionally include a tocopherol polyethylene glycol succinate (e.g., TPGS or vitamin E TPGS). The tocopherol polyethylene glycol is included in the formulation to enhance the thermal stability of the formulation, which in turn, can increase the formulation's shelf-life, which is particularly important in tropical regions of the world where prolonged exposure to high temperatures are common and refrigerated medicinal storage is rare. For formulations in which enhanced thermal stability is desired, the formulation includes a tocopherol polyethylene glycol succinate.

Structurally, tocopherol polyethylene glycol succinates have a polyethylene glycol (PEG) covalently coupled to tocopherol (e.g., a-tocopherol or vitamin E) through a succinate linker. Because PEG is a polymer, a variety of polymer molecular weights can be used to prepare the TPGS. In one embodiment, the TPGS is tocopherol polyethylene glycol succinate 1000, in which the average molecular weight of the PEG is 1000. One suitable tocopherol polyethylene glycol succinate is vitamin E TPGS commercially available from Eastman.

As used herein, “vitamin E” refers to a family of compounds that includes α-, β-, γ-, and δ-tocopherols and the corresponding tocotrienols.

The preparation of representative AmpB and protease inhibitor formulations of the invention that include fatty acid glycerol esters, polyethylene oxide-containing fatty acid esters, and a tocopherol polyethylene glycol succinate is based on the procedure described in PCT Publication No. WO2011/050457 for AmpB formulations.

In one aspect, the invention provides oral formulations of AmpB and protease inhibitor that are stable at the temperatures of WHO Climatic Zones 3 and 4 (30-43° C.). Four representative ampB and protease inhibitor formulations comprising mono- and di-glycerides (Peceol), pegylated esters (Gelucire 44/14), and optionally a vitamin E-TPGS (TPGS) are summarized in Table 2.

TABLE 3
Compositions of Representative
Protease Inhibitor Formulations

| Formu- | AmpB | Protease | Peceol/Gelucire | TPGS |
|---|---|---|---|---|
| lation | (mg/mL) | Inhibitor | 44/14 (v/v) | (v/v) |
| A | 5 | variable | 50:50 | 5 |
| B | 5 | variable | 60:40 | 5 |
| C | 5 | variable | 50:50 | 0 |
| D | 5 | variable | 60:40 | 0 |

In certain aspects, the present invention provides protease inhibitor formulations that can be orally administered. The protease inhibitor formulations of the invention provide excellent drug solubilization, drug stability in simulated gastric and intestinal fluids, and have significant activity.

The amphotericin B and/or protease inhibitor formulations of the invention can be self-emulsifying drug delivery systems. Self-emulsifying drug delivery systems (SEDDS) are isotropic mixtures of oils, surfactants, solvents, and co-solvents/surfactants. SEDDS can be used for the design of formulations in order to improve the oral absorption of highly lipophilic drug compounds, such as amphotericin B. When a SEDDS composition is released into the lumen of the gut, the composition disperses to form a fine emulsion, so that the drug remains in solution in the gut, avoiding the dissolution step that frequently limits the rate of absorption of hydrophobic drugs from the crystalline state. The use of SEDDS usually leads to improved bioavailability and/or a more consistent temporal profile of absorption from the gut. A description of compositions of SEDDS can be found in C. W. Pouton, Advanced Drug Delivery Reviews 25: 47-58 (1997).

The amphotericin B and/or protease inhibitor formulations of the invention can be orally administered in soft or hard gelatin capsules and form fine relatively stable oil-in-water (o/w) emulsions upon aqueous dilution owing to the gentle agitation of the gastrointestinal fluids. The efficiency of oral absorption of the drug compound from the SEDDS depends on many formulation-related parameters, such as the formulations' components, polarity of the emulsion, droplet size and charge, all of which in essence determine the self-emulsification ability. Thus, only very specific pharmaceutical excipient combinations will lead to efficient self-emulsifying systems.

In certain embodiments, any of the AmpB and/or protease inhibitor formulations of the present invention may further comprise one or more additional therapeutic agents.

In certain embodiments, the AmpB and/or protease inhibitor formulations further comprise one or more additional components of HAART. The usual HAART regimen combines three or more different drugs such as two nucleoside reverse transcriptase inhibitors (NRTIs) and a protease inhibitor (PI); two NRTIs and a non-nucleoside reverse transcriptase inhibitor (NNRTI); one NRTI, one PI and one NNRTI; or other such combination. In certain embodiments, it includes efavirenz, teofovir, and emtricitabine; rilpivirine, tenofovir, and emtricitabine; elvitegravir, cobicistat, tenofovir, and emtricitabine; dolutegravir, abacavir, and lamivudine; or tenofovir disoproxil fumarate (TDF)/emtricitabine (FTC), efavirenz, atazanavir/ritonavir, and darunavir.

Methods for Treatment with Amphotericin B

In certain embodiments, amphotericin B (AmpB) formulations of the present invention, including those that do or do not also comprise one or more protease inhibitors, are used to treat or prevent an infectious disease in a subject in need thereof. Thus, in certain aspects, the present invention provides a method for treating an infectious disease treatable by the administration of an amphotericin B formulation of the present invention. In particular embodiments, the infectious disease is human immunodeficiency virus (HIV), e.g., HIV-1. In particular embodiments, the subject has been diagnosed as being infected with HIV, e.g., HIV-1. In certain embodiments, the subject has been diagnosed with acquired immune deficiency syndrome (AIDS).

In a related embodiment, the present invention comprises a method of activating a latent HIV reservoir in a subject infected with HIV, comprising providing to the subject an AmpB formulation described herein, including those that do or not also comprise one or more protease inhibitors. In particular embodiments, the subject comprises latent HIV in memory CD4+ T cells and/or monocytes. In particular embodiments, the method activates latent HIV present in memory CD4+ T cells and/or monocytes. In particular embodiments, the method comprises reactivating HIV-infected CD4+ T cells and/or monocytes in the subject. In particular embodiments, the HIV is HIV-1. In certain embodiments, activating the latent HIV reservoir comprises inducing HIV production.

In a further related embodiment, the present invention comprises a method of eliminating HIV-infected CD4+ T cells and/or monocytes from a subject infected with HIV, comprising providing to the subject an AmpB formulation described herein, including those that do or do not also comprise one or more protease inhibitors. In particular embodiments, the HIV is HIV-1. In particular embodiments, the HIV-infected CD4+ T cells and/or monocytes are latently infected with HIV, e.g., HIV-1.

According to various aspects of the methods of the present invention, a therapeutically effective amount of an amphotericin B formulation of the invention, including those that do or do not also comprise one or more protease inhibitors, is administered to a subject in need thereof. In one embodiment, the formulation is administered orally. In another embodiment, the formulation is administered topically.

As used herein, the terms “treating” and “treatment” refer to reduction in severity and/or frequency of symptoms, elimination of symptoms and/or underlying cause, reduction in likelihood of the occurrence of symptoms and/or underlying cause, and improvement or remediation of damage. Thus, “treating” a patient with an active agent as provided herein includes prevention of a particular condition, disease or disorder in a susceptible individual as well as treatment of a clinically symptomatic individual. As used herein, “effective amount” refers to an amount covering both therapeutically effective amounts and prophylactically effective amounts. As used herein, “therapeutically effective amount” refers to an amount that is effective to achieve the desired therapeutic result. A therapeutically effective amount of a given active agent will typically vary with respect to factors such as the type and severity of the disorder or disease being treated and the age, gender, and weight of the patient.

In particular embodiments, a therapeutically effective amount of AmpB is an amount sufficient to achieve a blood plasma level of 0.01 uM to 10 mM, 0.01 uM to 1 mM, 0.01 uM to 100 nM, or 0.01 uM to 10 mM. In certain embodiments, a subject is provided with about 0.01 to about 1000 mg/kg, about 0.1 to about 100 mg/kg, about 0.5 to about 50 mg/kg, about 1 to about 20 mg/kg or about 5 to about 10 mg/kg, e.g., about 5, about 6, about 7, about 8, about 9, or about 10 mg/kg.

In particular embodiments, the subject is provided with an AmpB formulation described herein one or more, two or more, three or more, four or more, five or more, or six or more times, with a duration of time occurring between each provision. In particular embodiments, the subject is provided with the AmpB formulation once, twice, three times, four times, five times, six times, seven times, eight times, nine times, or ten times, with a duration of time between each provision. In particular embodiments, a subject is provided with the AmpB formulation about once per day for about four days, about once per day for about five days, about once per day for about six days, or about once per day for about one week. In particular embodiments, a subject is provided with the AmpB formulation once a day, twice a day, three times a day or four times a day, e.g., for any of the durations of time described here. In particular embodiments, the subject is provided with the AmpB formulation about once a day, twice a day, three times a day, four times a day, or once every two days for about three days, four days, five days six days, one week, two weeks, three weeks, one month or two months, or longer. In particular embodiments, the days and/or weeks are consecutive.

In further embodiments, a subject is provided with one or more additional therapeutic agents in combination with an ampB formulation of the present invention. In particular embodiments, the one or more additional therapeutic agent is provided to the subject at the same time as, before, or after the subject is provided with the AmpB formulation.

In certain embodiments, the one or more additional therapeutic agents comprises an agent used to treat HIV, e.g., HIV-1, infection in a subject, such as a nucleoside reverse transcriptase inhibitor (NRTI), a protease inhibitor (PI), or a non-nucleoside reverse transcriptase inhibitor (NNRTI). In one embodiment, the one or more additional therapeutic agents comprise a highly active antiretroviral therapy (HAART). HAART is an aggressive treatment regimen used to suppress HIV viral replication and progression of HIV. The usual HAART regimen combines three or more different drugs such as two nucleoside reverse transcriptase inhibitors (NRTIs) and a protease inhibitor (PI); two NRTIs and a non-nucleoside reverse transcriptase inhibitor (NNRTI); one NRTI, one PI and one NNRTI; or other such combination. In particular embodiments, an AmpB formulation of the present invention is provided to the subject in combination with a protease inhibitor formulation of the present invention.

In other embodiments, the additional therapeutic agent comprises an immune modulating agent, such as, e.g., anti-PD-1 or Ipilimumab (anti-CTLA-4). In particular embodiments, treatment with a combination of an AmpB formulation of the present invention and the immune modulating agent synergistically activates latently infected memory CD4+ T cells.

Methods for Treatment with Protease Inhibitors

In certain embodiments, protease inhibitor formulations of the present invention, including those that do or do not also include AmpB, are used to treat or prevent an infectious disease in a subject in need thereof. Thus, in certain aspects, the present invention provides a method for treating an infectious disease treatable by the administration of a protease inhibitor. In particular embodiments, the infectious disease is human immunodeficiency virus (HIV), e.g., HIV-1. In particular embodiments, the subject has been diagnosed as being infected with HIV, e.g., HIV-1. In certain embodiments, the subject has been diagnosed with acquired immune deficiency syndrome (AIDS). In other embodiments, the disease is Hepatitis C. In other embodiments, protease inhibitor formulations of the present invention, including those that do or do not also include AmpB, are used to treat or prevent a protozoal infection in a subject in need thereof, e.g., malaria or Chagas disease; gastrointestinal infections, e.g., Giardia; or cancer.

Protease inhibitors have a number of side effects, such as lipodystrophy, hyperlipidemia, diabetes mellitus type 2, and kidney stones. In certain embodiments, the present invention provides methods to reduce one or more of these side effects, comprising providing a protease inhibitor in a formulation of the present invention to a subject in need thereof.

HIV-1 replication has been associated with lower antiretroviral drug concentrations in lymphatic tissues, probably due to it not getting to these tissues in sufficient amounts from systemic blood circulation. In certain embodiments, the present invention provides methods to enhance or increase the delivery of a protease inhibitor to a subject in need thereof s lymphatic tissues, comprising providing the protease inhibitor in a formulation of the present invention to the subject in need thereof.

Protease inhibitors exhibit low brain permeability. As a result, unchallenged HIV viral replication can lead to HIV encephalitis and antiretroviral drug resistance. For example, indinavir is an anti-retroviral protease inhibitor used as a part of the HAART regimen is some patients with AIDS. However, the sub-therapeutic concentration of indinavir in the brain leads to failure of treatment and results in the development of drug-resistant viral strains in the brain despite the presence of adequate plasma concentrations. In certain embodiments, the present invention provides methods to enhance or increase the delivery of a protease inhibitor to a subject in need thereof's brain (e.g., increase the brain concentration of a protease inhibitor), comprising providing the protease inhibitor in a formulation of the present invention to the subject in need thereof. In another embodiment, the present invention provides methods to reduce or inhibit the development of resistance to a protease inhibitor in a subject, comprising providing the protease inhibitor in a formulation of the present invention to a subject in need thereof. In a related embodiment, the present invention provides methods to enhance or increase permeation of the blood brain barrier by a protease inhibitor, comprising providing the protease inhibitor in a formulation of the present invention to a subject in need thereof. In certain embodiments, the present invention provides methods to decrease HIV viral load in a subject in need thereof's brain, comprising providing the protease inhibitor in a formulation of the present invention to the subject in need thereof. In particular embodiments, the protease inhibitor is atazanavir or indinavir.

According to various aspects of the methods of the present invention, a therapeutically effective amount of a protease inhibitor formulation of the invention, including those that do or do not also include AmpB, is administered to a subject in need thereof. In one embodiment, the formulation is administered orally. In another embodiment, the formulation is administered topically. As used herein, the terms “treating” and “treatment” refer to reduction in severity and/or frequency of symptoms, elimination of symptoms and/or underlying cause, reduction in likelihood of the occurrence of symptoms and/or underlying cause, and improvement or remediation of damage. Thus, “treating” a patient with an active agent as provided herein includes prevention of a particular condition, disease or disorder in a susceptible individual as well as treatment of a clinically symptomatic individual. As used herein, “effective amount” refers to an amount covering both therapeutically effective amounts and prophylactically effective amounts. As used herein, “therapeutically effective amount” refers to an amount that is effective to achieve the desired therapeutic result. A therapeutically effective amount of a given active agent will typically vary with respect to factors such as the type and severity of the disorder or disease being treated and the age, gender, and weight of the patient.

In particular embodiments, a therapeutically effective amount of a protease inhibitor is an amount sufficient to achieve a therapeutically effective blood plasma level. Therapeutically effective dosages and blood plasma levels of protease inhibitor are known in the art. For example, in particular embodiments, tipranavir is administered at about 100-2000 mg (e.g., about 500 mg) twice per day; indinavir is administered at about 100-2000 mg (e.g., about 800 mg) every eight hours or twice per day; saquinavir is administered at about 100-5000 mg (e.g., about 1000 mg) twice per day; lopinavir is administered at about at about 100-2000 mg (e.g., about 800 mg) lopinavir per day; ritonavir is administered at about 100-2000 mg (e.g., 200 mg) per day; lopinavir and ritonavir are administered in combination at about 100-200 mg each (e.g., about 800 mg lopinavir and about 200 mg ritonavir) per day; fosamprenavir is administered at about 100-5000 mg (e.g., about 1400 mg) twice per day; ritonavir is administered at about 100-2000 mg (e.g., about 600 mg) twice per day; darunavir is administered at about 100-5000 mg (e.g., about 800 mg per day); atazanavir is administered at about 100-2000 mg (e.g., about 400 mg per day; bracanavir may be administered at about 50-800 mg twice per day; and nelfinavir (viracept) is administered at about 100-5000 mg (e.g., about 1300 mg) twice per day. In certain embodiments, the blood plasma level is 0.01 uM to 10 mM, 0.01 uM to 1 mM, 0.01 uM to 100 nM, or 0.01 uM to 10 mM.

In particular embodiments, the subject is provided with a protease inhibitor formulation described herein one or more, two or more, three or more, four or more, five or more, or six or more times, with a duration of time occurring between each provision. In particular embodiments, the subject is provided with the protease inhibitor formulation once, twice, three times, four times, five times, six times, seven times, eight times, nine times, or ten times, with a duration of time between each provision. In particular embodiments, a subject is provided with the protease inhibitor formulation about once per day for about four days, about once per day for about five days, about once per day for about six days, or about once per day for about one week. In particular embodiments, the subject is provided with the protease inhibitor formulation about once a day, twice a day, three times a day, four times a day, or once every two days. In certain embodiments, the subject is provided with the protease inhibitor formulation from one to four times per day. In certain embodiments, the subject is provided with the protease inhibitor formulation for about three days, four days, five days six days, one week, two weeks, three weeks, one month or two months, or longer. In particular embodiments, the days and/or weeks are consecutive. In certain embodiments, the subject is provided with the protease inhibitor formulation for at least four months, at least six months, at least one year, at least two years, or longer.

In further embodiments, a subject is provided with one or more additional therapeutic agents in combination with a protease inhibitor formulation of the present invention. In particular embodiments, the one or more additional therapeutic agent is provided to the subject at the same time as, before, or after the subject is provided with the protease inhibitor formulation. In certain embodiments, the one or more additional therapeutic agent(s) is present in the same formulation as the protease inhibitor. In certain embodiments, the additional therapeutic agent is AmpB, and in particular embodiments, the additional therapeutic agent is an AmpB formulation of the present invention.

In one embodiment, the one or more additional therapeutic agents comprise a highly active antiretroviral therapy (HAART). HAART is an aggressive treatment regimen used to suppress HIV viral replication and progression of HIV. The usual HAART regimen combines three or more different drugs such as two nucleoside reverse transcriptase inhibitors (NRTIs) and a protease inhibitor (PI); two NRTIs and a non-nucleoside reverse transcriptase inhibitor (NNRTI); one NRTI, one PI and one NNRTI; or other such combination. In certain embodiments, the protease inhibitor component of HAART is provided to a subject in a formulation of the present invention. In certain embodiments, one or more additional components of HAART are provided to the subject in the same formulation.

In certain embodiments, a protease inhibitor formulation of the present invention is provided to a subject in need thereof in combination with ritonavir, e.g., to boost blood levels of the protease inhibitor and extend dosing intervals.

In certain embodiments, a protease inhibitor formulation of the present invention is provided to a subject in need thereof in combination with an inhibitor of P-glycoprotein, e.g., to inhibit the efflux by P-glycoprotein of the protease inhibitor from the brain.

### Example 1

### Reactivation of Latent HIV Reservoirs Using AmpB Formulations

This study evaluates that ability of an Amphotericin B formulation of the present invention (oral Amp-B) to reactivate and eliminate latently infected CD4+ T cells and monocytes in order to purge the latent human immunodeficiency virus (HIV) reservoir, e.g., when used in combination with highly active antiretroviral therapy (HAART). A flowchart of the study design is provided in FIG. 1.

### Subjects and Studies

Eight (8) HIV-infected subjects successfully treated with HAART and harboring a latent viral reservoir were recruited. HIV-1 infection was detected by ELISA and confirmed by Western blot for p24 antigen. HAART regimen consisted of the following: two nucleoside reverse transcriptase inhibitors (NRTI), and a non-nucleoside reverse transcriptase inhibitor (NNRTI) and/or at least one protease inhibitor (PI). Subjects receiving one NRTI, one NNRTI, and one PI regimen also met the study criteria. All study participants were chronically infected and started HAART at a nadir CD4+ T cell count of ≤300 cells/uL and a CD4/CD8 T cell ratio of <1.

Leukapheresis samples were collected from these subjects and used in several virologic and immunologic assays in order to evaluate the impact of oral Amp-B on the reactivation and the elimination of latent HIV-1 reservoir in vitro. These assays included PBMC isolation, Human Leukocyte Antigen (HLA) Class I-typing to determine potential peptide MHC Class I multimers used to detect HIV-specific CD8+ T cells, generation of monocyte-derived dendritic cells, T cell and monocyte phenotyping, purification of CD14+ monocytes and memory CD4+ T cells by magnetic-activated sorting (MACS) followed by lysis and quantification of the size of the latent HIV reservoir, co-culturing of MACS-purified CD4 memory T cells and CD14+ monocytes for six days in the presence or absence of three different concentration of oral Amp-B to quantify reactivation of the latent HIV reservoir by qRT-PCR or by p24 enzyme linked immunosorbent assay (ELISA) in cell culture supernatants, quantification of the residual HIV reservoir, and co-culturing of carboxy-fluorescein diacetate succinidimidyl ester (CFSE) labeled total PBMC and magnetic activated cell sorting (MACS)-sorted memory CD4+/total CD8+ T cells for six days in the presence or absence of three different concentrations of oral Amp-B followed by intracellular cytokine staining (ICS) in order to define the effect of the oral Amp-B on the immune response.

Oral Amp-B was used at three different concentrations: 0.04 uM, 0.2 uM, and 1 uM. The stock oral Amp-B formulation included Amp-B (4650 ug/mL) in PECEOL®/GELUCIRE® 44/14 (50:50 v/v)+5% (v/v) Vitamin E-TPGS. Amphotericin B (from Streptomyces sp., Calbiochem, >80% purity) was purchased from Sigma (St. Louis Mo.). PECEOL® (glyceryl oleate) and GELUCIRE® 44/14 were obtained from Gattefosse Canada (Mississauga, Ontario). D-alpha-tocopheryl polyethylene glycol succinate (Vitamin E-TPGS; NF grade) was purchased from Eastman Chemical Co. (Kingsport, Tenn.), and typically contained 260-300 mg/g vitamin E as d-α-tocopherol. The formulations were prepared as described in U.S. Pat. No. 8,673,866.

### Quantification of Proviral HIV DNA Burden in Cell Subsets

The size of the reservoir in different subsets of primary immune cells (CD14+ monocytes, peripheral blood mononuclear cells (PBMC), CD8+ and memory CD4+ T cells) obtained from the subjects was measured, and the memory CD4+ T cell compartment was identified as the main reservoir of latent HIV. CD14+ monocytes, total CD8+ and memory CD4+ T cells were isolated from PBMC using an optimized immunomagnetic bead-based negative selection protocol. The purity of each sorted cell population was assessed by flow cytometry and consistently exceeded 80%. To determine the frequency of PBMC, CD14+ monocytes, total CD8+ and memory CD4+ T cells carrying HIV provirus in infected individuals, cell lysates were used in a nested Alu PCR to quantify both integrated HIV DNA and CD3 gene copy numbers.

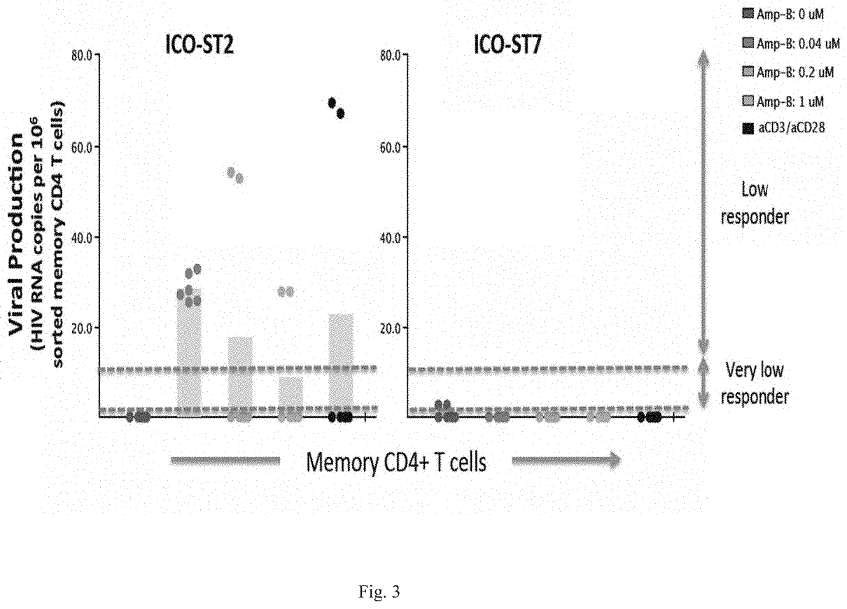

Levels of proviral DNA in PBMC, CD8+ T cells, memory CD4+ T cells, and CD14+ monocytes were determined (detection limits=3 copies/10⁶ cells) for all subjects and is shown in FIG. 3. Latently infected cells were detectable in all individuals studied except one, confirming the validity of the subject selection criteria. In agreement with prior studies, HIV DNA levels were higher in sorted memory CD4+ T cells (median of 525 HIV DNA copies per 10⁶ cells) compared to PBMC (median of 73 HIV DNA copies per 10⁶ cells), identifying the memory CD4+ T cell compartment as the main viral reservoir in seropositive subjects. Proviral DNA was not detectable or below the limit of detection of this assay in CD8+ T cells for all subjects. The level of HIV-1 proviral DNA in sorted CD14+ monocytes was only detectable in two subjects. Of note, in one of these subjects, proviral DNA level was very low and only found in one of three biological replicates. The inability to detect HIV proviral DNA in circulating monocytes can be due to the limit of detection of the assay but most probably can be explained either by the duration of HAART treatment or that infected monocytes already migrated into tissue.

### Effect of Oral-AmpB on Viral Production in CD4+ T Cells and CD14+ Monocytes

To determine whether oral Amp-B induced viral production in highly purified and latently infected memory CD4+ T cells and monocytes isolated from virally suppressed subjects, an ultrasensitive assay was used to measure viral release from primary cells following stimulation. The average purity of the memory CD4+ T cells and CD14+ monocytes after sorting exceeded 95% and 85%, respectively.

To assess HIV reactivation by oral Amp-B, 3×10⁶ memory CD4+ T cells or CD14+ monocytes were plated in a 96 deep well plate with 1 mL of RPMI supplemented with 10% HS and in increasing concentrations of oral Amp-B (0.04, 0.2 and 1 uM). Anti-CD3/anti-CD28 antibody-mediated activation of cells was used as a positive control. In order to prevent new rounds of infection, anti-retroviral drugs (ARVs; 180 nM Azidovudine, 100 nM Efavirenz and 200 nM Raltegravir) were added on day 1 and day 4. All conditions were performed in triplicate. On day 6, culture supernatants or cells were harvested for the analysis of HIV RNA or the quantification of remaining proviral HIV DNA. Viral release for each experimental condition was measured in duplicate by ultrasensitive RT-PCR with a detection limit of a single copy of HIV RNA using 1 mL of culture supernatant. HIV-infected subjects were defined as very low or low responders when viral production was >1 or >10 HIV RNA copies per 10⁶ sorted memory CD4 T cells, respectively. HIV-infected subjects were defined as non-responder when viral production was under the assay detection limit (<1 HIV RNA copies per 10⁶ sorted memory CD4 T cells).

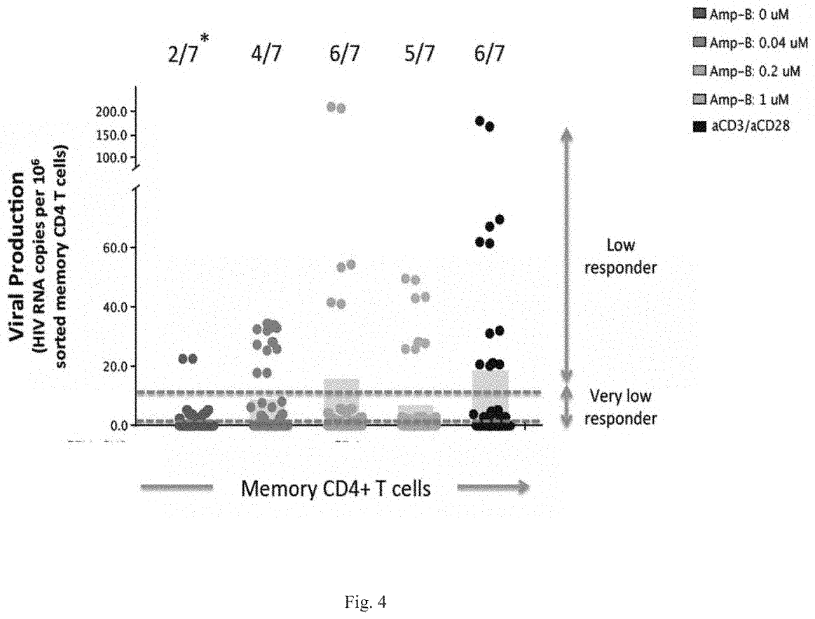

A subset of subjects showed low levels of viral production (>10 HIV RNA copies per 10⁶ sorted memory CD4 T cells) but comparable to data published on the effect of IL-7 or SAHA using the same method and conditions to measuring the HIV reservoirs (purified CD4+ T cells, co-culture in the presence of HAART, ultrasensitive RT-PCR). FIG. 3 shows a representative example of two subjects, an oral Amp-responder (ICO-ST2, viral production>10 HIV RNA copies per 10⁶ sorted memory T cells) and a non-responder (ICO-ST7, <1 HIV RNA copies per 10⁶ sorted memory T cells) as measured by HIV reactivation from memory CD4+ T cells. Most supernatants collected from cultured memory CD4+ T cells in the absence of oral Amp-B did not produce or produced very low levels of virus (with the exception of one replicate in one subject), whereas viral production was readily detected following αCD3/αCD28 stimulation of these samples (FIG. 4). Interestingly, a subset of subjects showed detectable levels of viral production (>10 HIV RNA copies per mL) in the presence of oral Amp-B. However, these levels of viral production were not generally reproducible among the triplicates. At the concentration of 0.2 uM of oral Amp-B, viral production in memory CD4+ T cells was induced in six of seven subjects tested, although the levels of viral production were not reproducible when compared to those obtained with 0.04 uM of oral Amp-B (FIG. 4).

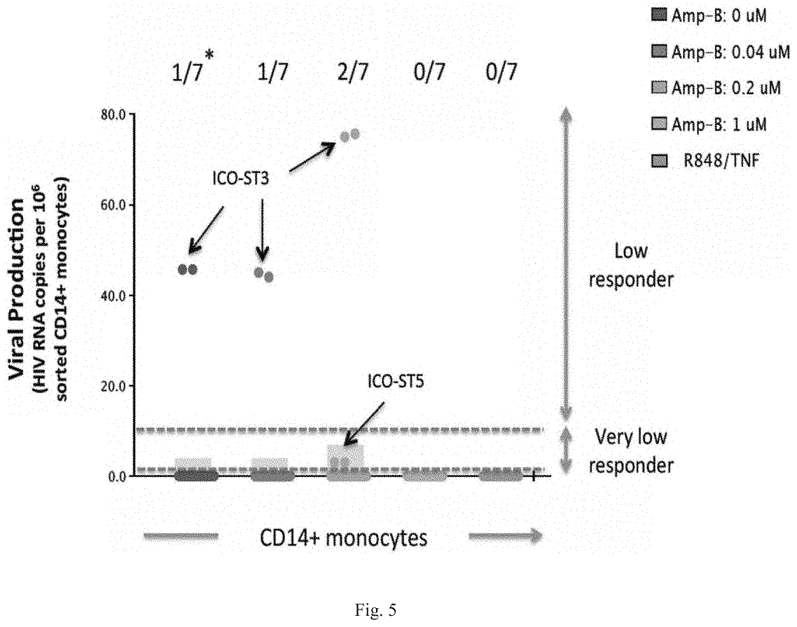

Cultures containing R848 and TNF were used as positive controls for the stimulation and viral reactivation in CD14+ monocytes. Under these potent activation conditions, viral production was undetectable (FIG. 5). Oral Amp-B did not yield detectable viral production, except for 3 independent cultures tested in duplicate. This was probably due to contaminating CD4+ T cells and a similar signal was detected in samples not containing oral Amp-B. Based on these results, circulating CD14+ monocytes do not harbor any integrated HIV latent reservoir consistent with the failure of oral Amp-B to reactivate viral production. Thus, these results showed that among circulating target cells, monocytes contain a minimal amount of detectable proviral HIV DNA compared to memory CD4+ T cells in the subjects recruited to this study.

### Effect of Oral AmpB on the Size of the Viral Reservoir

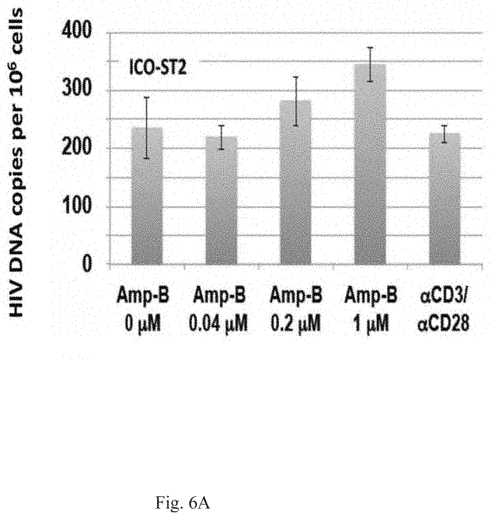

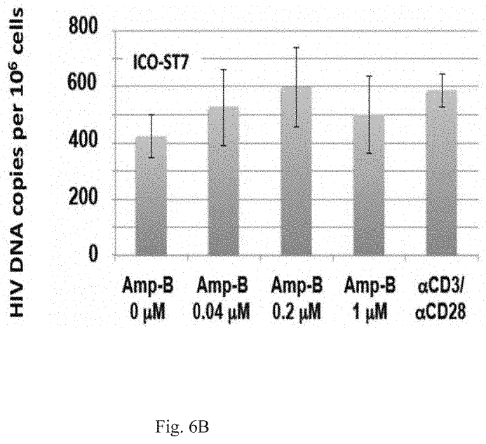

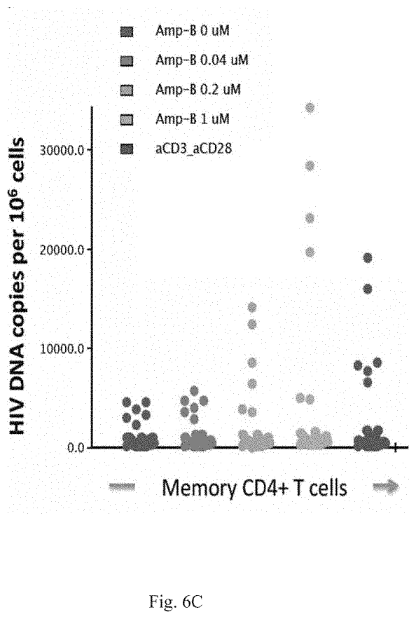

To assess the impact of oral Amp-B on latently infected memory CD4+ T cells, pellets collected from cultured highly-purified memory CD4+ T cells in the absence or presence of different concentrations of oral Amp-B were used to quantify the size of the latent reservoir after 6 days of in vitro activation. Although oral Amp-B induced viral production in memory CD4+ T cells from 6 of 7 subjects tested (FIG. 4), it did not reduce HIV DNA levels in latently infected memory CD4+ T cells (FIG. 6). Moreover, increasing the concentration of oral Amp-B was associated with a concomitant increase in the size of the reservoir. This may be due to the toxicity of the product or to transient proliferation of latently infected CD4+ T cells during the 6 day co-culture.

### Effect of Oral AmbB on CD4+ and CD8+ T Cell Counts Following Activation

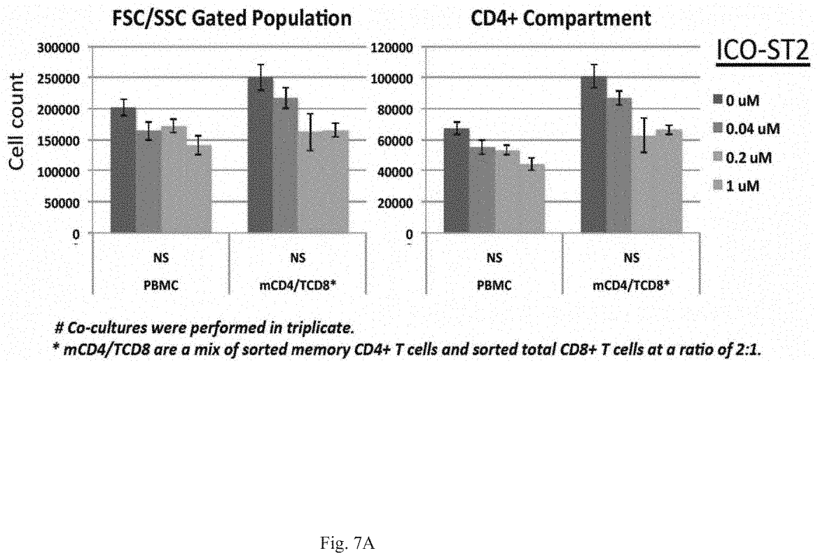

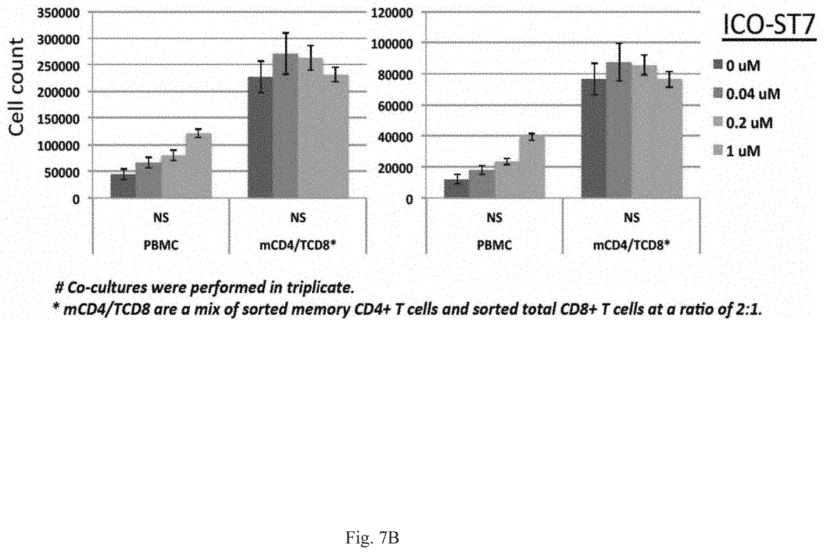

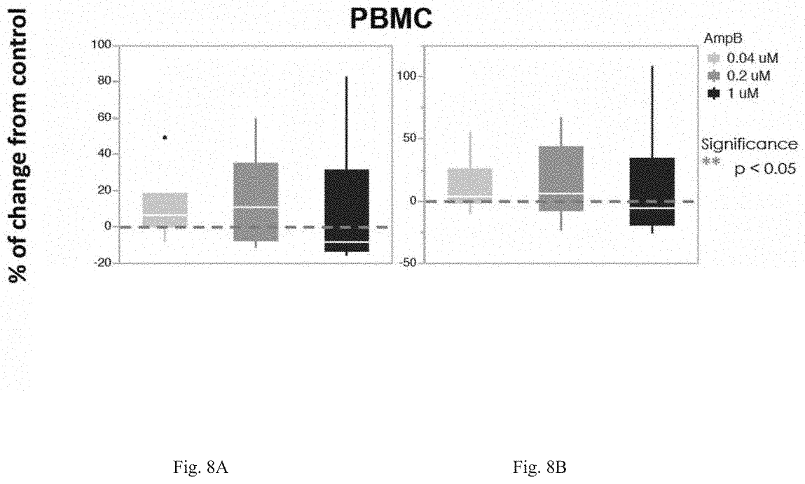

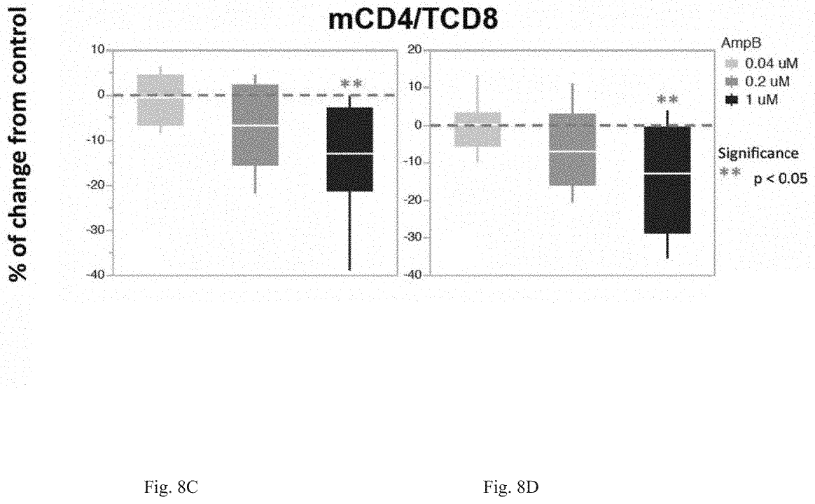

The effect of oral Amp-B on T cell counts following activation was determined. Increasing concentrations of oral Amp-B was associated with a marked decrease in the cell count in all cellular compartments analyzed (PBMC or sorted memory CD4+ T cells/total CD8+ T cells) in one subject (FIG. 7A). In stark contrast, cell counts increase in a dose-dependent fashion in another subject in both the FSC/SSC gated population and the CD4+ T cell compartment in PBMC following stimulation. However, no oral Amp-B-mediated effect on cell counts was detected in memory CD4+ T cell/total CD8+ T cell co-cultures following stimulation (FIG. 7B). The entire cohort was examined and the % change in cellular counts in the presence of Amp-B from the control condition (0 uM oral Amp-B) was determined. Wilcoxon Signed-Rank tests were performed. The results of these analyses are summarized in FIG. 8, and show that oral Amp-B had no effect on the CD4+ or CD8+ T cell count in total PBMC (FIG. 8, Top panel). However, increasing the concentration of oral Amp-B had an adverse effect on the cell counts in both CD4+ and CD8+ compartments in sorted memory CD4+ T cells/total CD8+ T cells. The percent change was significantly different in the 1 uM versus 0 uM condition confirming a clear reduction in cellular counts in the presence of 1 uM of Amp-B (FIG. 8, Bottom panel).

### Phenotype of Co-Cultured CD4+ and CD8+ T Cells in the Presence of Oral AmpB

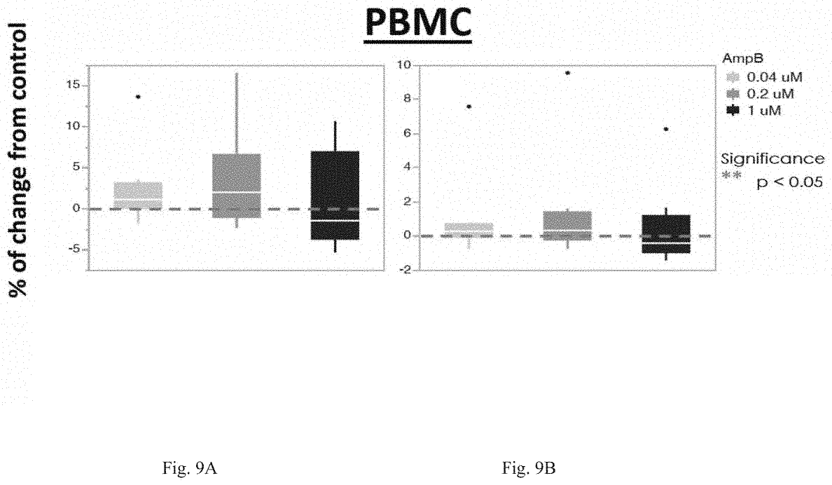

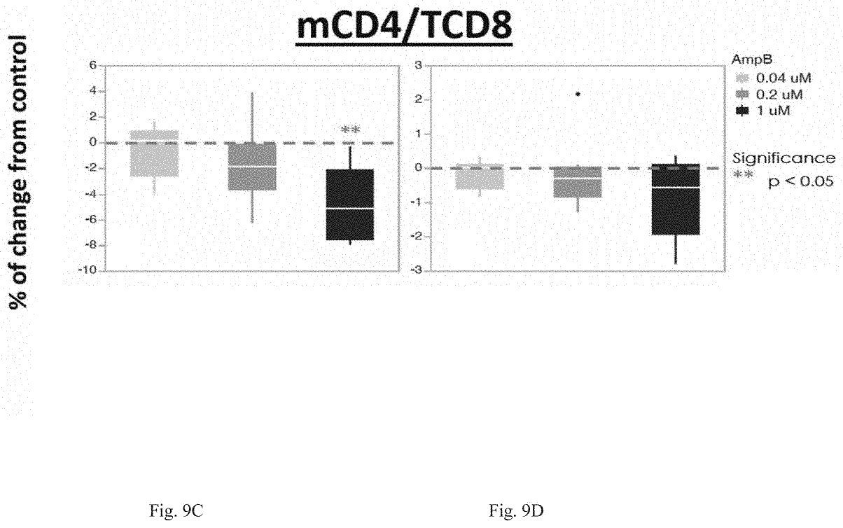

In order to determine whether oral Amp-B modified the composition of the memory CD4+ T cell compartment (known to harbor latent HIV reservoir), the phenotypes of both CD4+ and CD8+ T cells in unstimulated PBMC or sorted memory CD4+/Total CD8+ T cells in the presence of different concentrations of oral Amp-B was characterized. For one subject (a virological responder), increasing doses of oral Amp-B induced a significant reduction in the frequency of the CD45RA−CCR7+CD27+CM CD4+ T cells in whole PBMC, and this effect was more pronounced on sorted memory CD4+ T cells (data not shown). A concomitant increase in the frequency of CD45RA−CCR7−CD27−EM CD4+ T cells was also observed in this subject (data not shown). In contrast, increasing the concentration of oral Amp-B in another subject (virological non-responder) did not have a pronounced effect on the frequencies of the CD45RA−CCR7+CD27+CM CD4+ T cells in PBMC or in sorted memory CD4+ T cells/CD8+ T cells (data not shown). When analyzing the entire cohort, the percent reduction in CD45RA−CCR7+CD27+CM cells was significant at the highest concentration of oral Amp-B (FIG. 9) and seemed to affect preferentially CD4+ T cells (FIG. 9, Bottom panel). Overall, increasing the concentration of oral Amp-B induced a shift towards larger cell counts of both naive CD4+ and CD8+ T cells in sorted memory CD4+/total CD8+ T cell populations (but not in whole PBMC).

### Effect of Oral AmpB on PD-1 or CTLA-4 Expression in CD4+ and CD8+ T Cells

Expression of the activation/exhaustion marker PD-1 was analyzed in the virological responder subject, in order to confirm whether the reactivation of the reservoir detected in this individual was associated with decreased expression of PD-1 on central memory (CM) and/or transitional memory (TM) CD4+ T cells. The addition of oral Amp-B at 0.04 uM and 0.2 uM slightly reduced the frequency of TM CD4+ T cells expressing PD-1 in sorted memory CD4+/total CD8+ T cells (data not shown). However, only the highest concentration of oral Amp-B (1 uM) reduced the frequency of PD-1-expressing central memory CD4+ T cells. In contrast, oral Amp-B did not impact the frequency of CM CD4+ T cells expressing PD-1 in the non-responder subject (data not shown). A trend towards increased frequencies of PD-1-expressing TM and EM CD4+ T cells in sorted memory CD4+/Total CD8+ T cells was observed (data not shown).

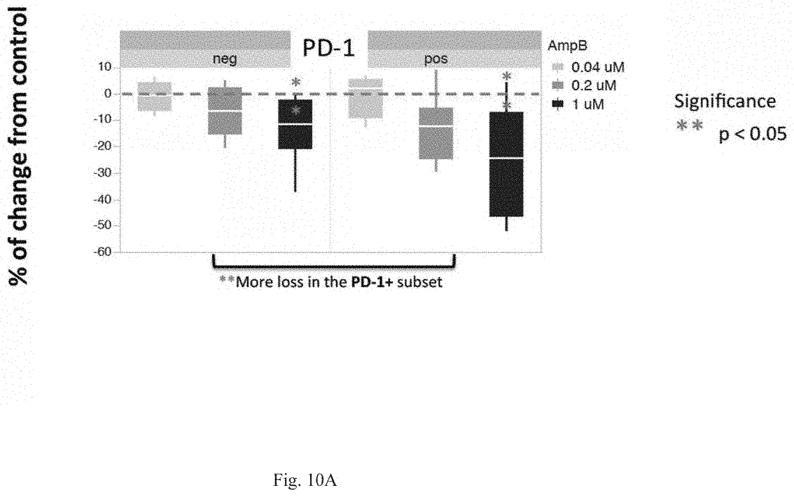

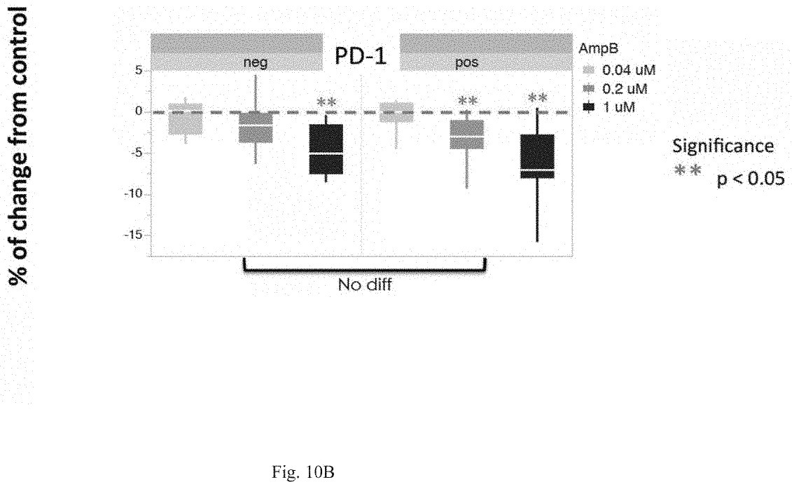

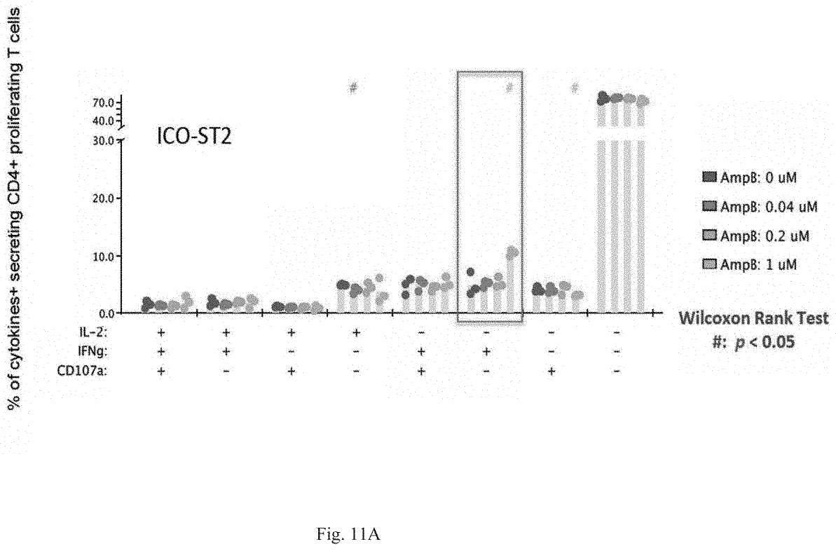

To evaluate the effect of oral Amp-B on PD-1+ and PD-1− populations among all study subjects, a statistical analysis was performed on four target populations: total CD4+, total CD8+, CM CD4+ and CM CD8+ T cells. As shown in FIG. 10, increasing the concentration of oral Amp-B reduced the cell counts of both CD4+ and CM CD4+ T cells irrespective of PD-1 expression. However, PD-1 expressing cells were more sensitive to lower doses of oral Amp-B relative to PD-1 negative cells when analyzing total CD4 T cells (FIG. 10A). Moreover, the negative effect of oral Amp-B on cell viability was also observed on CM CD8+ T cells preferentially expressing PD-1 (data not shown).

### Effect of Oral AmbB of the Polyfunctionality of Proliferative HIV-Specific T Cells

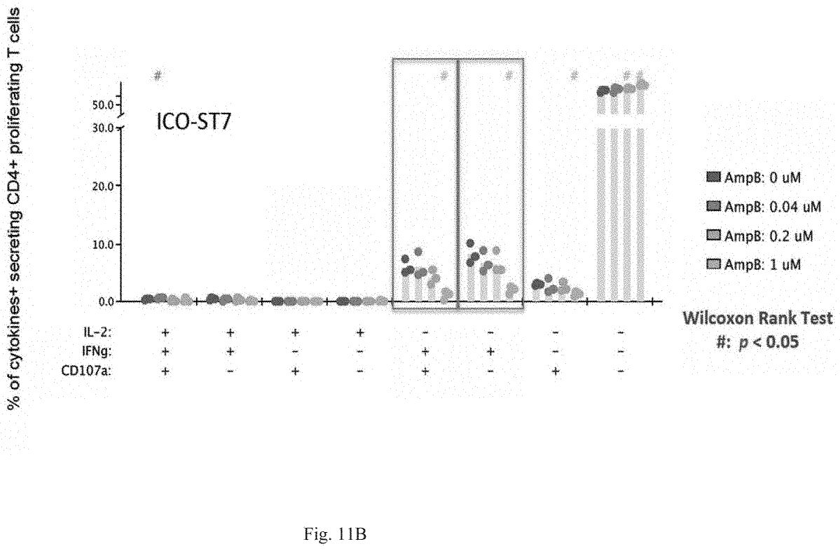

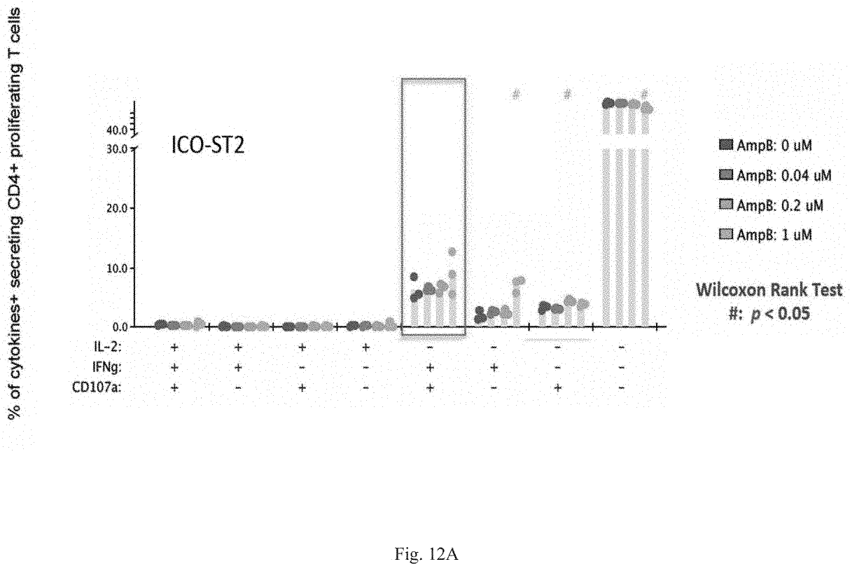

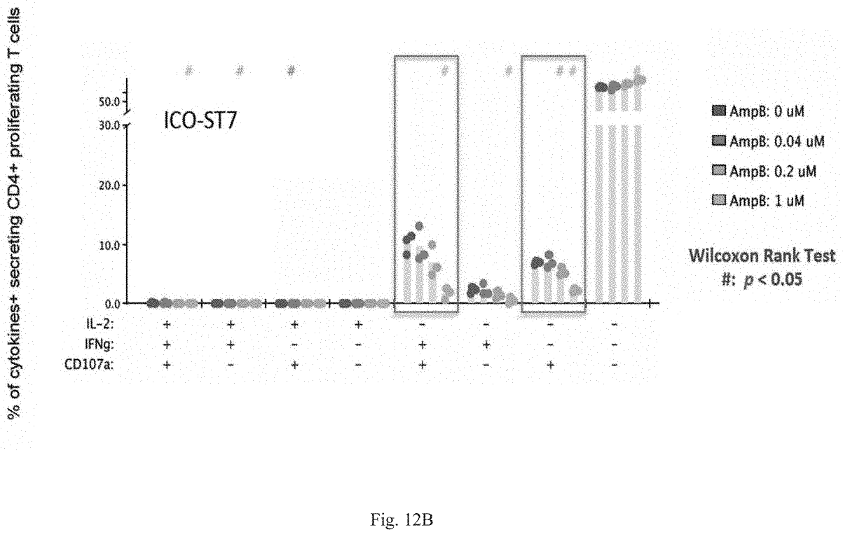

The effect of oral Amp-B on the proliferative and functional capacity of HIV-specific CD4+ and CD8+ T cells was analyzed. In the virological responder subject, only the highest concentration of oral Amp-B (1 uM) enhanced the proportion of monofunctional IFN-γ-secreting CD4+ and CD8+ T cells (FIGS. 11 and 12, top panel). However, the same concentration of oral Amp-B decreased the frequencies of both proliferating CD4+ and CD8+ T cells secreting IFN-γ and degranulating (CD107a+) in the non-responder (FIG. 12, bottom panel). Of note, increasing the dose of oral Amp-B did not significantly enhance the frequency of polyfunctional HIV-specific CD4+ T cells, nor HIV-specific CD8+ T cells in either PBMC or in sorted memory CD4+ T cells/total CD8+ T cells following 6 days of stimulation with HIV peptide pools.

### DISCUSSION

In this study, a cohort of 8 HIV-infected subjects was recruited and latent integrated HIV DNA in purified memory CD4+ T cells identified in seven of the eight subjects. The analysis of large numbers of highly purified memory CD4+ T cells from 7 HIV-infected subjects, demonstrated that oral Amp-B promotes the induction of viral production from latently infected cells in some subjects. The partial or total absence of viral production in the tested subjects may be due to the presence of a substantial HIV reservoir that is refractory to any stimulus in vitro. However, the reactivation of latent HIV by oral Amp-B or anti-CD3/anti-CD28 was not associated with partial or complete elimination of this reservoir.

In addition, it was demonstrated that increasing the concentration of oral Amp-B induced a significant reduction in the frequency of the central memory CD4+ T cells that harbor the HIV reservoir. Moreover, this phenomenon was inversely associated with a decreased expression of PD-1 in CD4+ T cells.

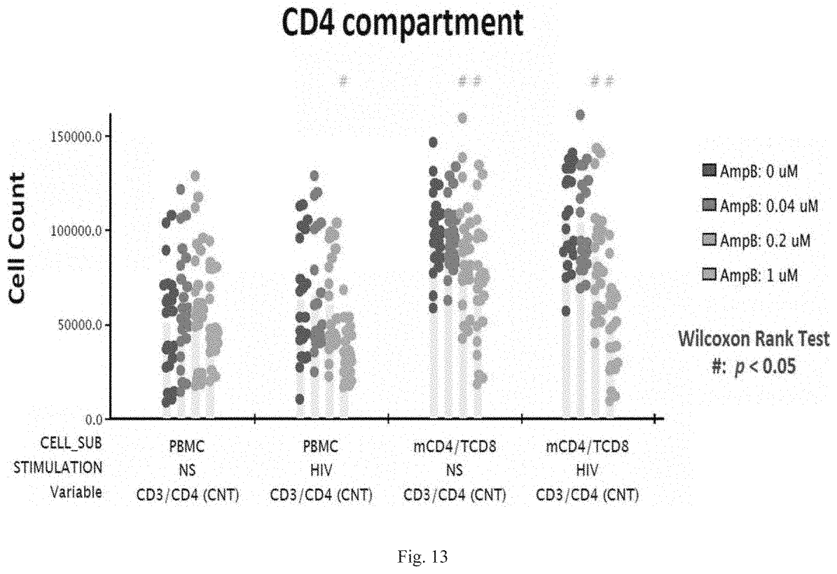

Importantly, it was shown that increasing the concentration of oral Amp-B decreased the number of cells after 6 days of stimulation for all subjects tested (whole PBMC and sorted cells). More importantly, this effect was more pronounced in the stimulatory conditions (FIG. 13). The latter observation did not enable a conclusion regarding whether oral Amp-B induced polyfunctionality or increased the proliferative capacity of HIV-specific CD8+ and CD4+ T cells.

This study suggests that Amp-B treatment can be used to reactivate a latent HIV reservoir. In addition, combining Amp-B with other immune modulating drugs such as anti-PD-1 and Ipilimumab (anti-CTLA-4) could synergistically activate latently infected memory CD4 T cells.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A protease inhibitor formulation, comprising:
(a) a protease inhibitor;
(b) one or more fatty acid glycerol esters;
(c) one or more polyethylene oxide-containing phospholipids or one or more polyethylene oxide-containing fatty acid esters; and
(d) optionally, a tocopherol polyethylene glycol succinate.

2. The protease inhibitor formulation of claim 1, further comprising:
(e) amphotericin B.

3. The protease inhibitor formulation of claim 1, comprising:
(a) a protease inhibitor;
(b) one or more fatty acid glycerol esters;
(c) one or more polyethylene oxide-containing phospholipids; and
(d) optionally, a tocopherol polyethylene glycol succinate.

4. The protease inhibitor formulation of claim 1, comprising:
(a) a protease inhibitor;
(b) one or more fatty acid glycerol esters;
(c) one or more polyethylene oxide-containing fatty acid esters; and
(d) optionally, a tocopherol polyethylene glycol succinate.

5. The protease inhibitor formulation of claim 1, wherein the formulation comprises the tocopherol polyethylene glycol succinate.

6. The protease inhibitor formulation of claim 5, wherein the tocopherol polyethylene glycol succinate is a vitamin E tocopherol polyethylene glycol succinate.

7. (canceled)

8. The protease inhibitor formulation of claim 1, wherein the protease inhibitor is selected from the group consisting of: amprenavir, ritonavir, saquinavir, tipranavir, atazanavir, fosamprenavir, lopinavir, indinavir, darunavir, and nelfinavir.

9.-25. (canceled)

26. A method of treating an infectious disease in a subject in need thereof, comprising providing to the subject the protease inhibitor formulation of claim 1.

27. The method of claim 26, wherein the protease inhibitor formulation is provided to the subject orally or topically.

28. The method of claim 26, wherein the infectious disease is human immunodeficiency virus type 1 (HIV-1) infection or acquired immune deficiency syndrome (AIDS).

29. The method of claim 26, wherein the infectious disease is a protozoal infection.

30.-33. (canceled)

34. A method of reactivating a latent HIV reservoir in a subject in need thereof, comprising providing to the subject an amphotericin B (AmpB) formulation comprising:
(a) AmpB;
(b) one or more fatty acid glycerol esters;
(c) one or more polyethylene oxide-containing phospholipids or one or more polyethylene oxide-containing fatty acid esters; and
(d) optionally, a tocopherol polyethylene glycol succinate.

35. The method of claim 34, wherein the AmpB formulation further comprises:
(e) a protease inhibitor.

36. The method of claim 34, wherein the AmpB formulation comprises:
(a) AmpB;
(b) one or more fatty acid glycerol esters;
(c) one or more polyethylene oxide-containing phospholipids; and
(d) optionally, a tocopherol polyethylene glycol succinate.

37. The method of claim 34, wherein the AmpB formulation comprises:
(a) AmpB;
(b) one or more fatty acid glycerol esters;
(c) one or more polyethylene oxide-containing fatty acid esters; and
(d) optionally, a tocopherol polyethylene glycol succinate.

38. The method of claim 34, wherein the formulation comprises the tocopherol polyethylene glycol succinate.

39. The method of claim 38, wherein the tocopherol polyethylene glycol succinate is a vitamin E tocopherol polyethylene glycol succinate.

40.-60. (canceled)

61. The method of claim 34, wherein the AmpB formulation is provided to the subject orally or topically.

62. The method of claim 34, wherein the subject has been diagnosed with latent human immunodeficiency virus type 1 (HIV-1) infection or acquired immune deficiency syndrome (AIDS).

63. The method of claim 34, wherein the AmpB formulation is provided to the subject at least once a day, at least once every two days, or at least once a week, for a period of time.
